(19) United States
(12) Patent Application Publication
(10) Pub. No.: US 2021/0277135 A1
(43) Pub. Date: Sep. 9, 2021

(54) OX-40 AGONIST, PD-1 PATHWAY INHIBITOR AND CTLA-4 INHIBITOR COMBINATION FOR USE IN A METHOD OF TREATING A CANCER OR A SOLID TUMOR

(71) Applicant: Bristol-Myers Squibb Company, Princeton, NJ (US)

(72) Inventors: Chan GAO, Redwood City, CA (US); Michael QUIGLEY, San Carlos, CA (US)

(73) Assignee: Bristol-Myers Squibb Company, Princeton, NJ (US)

(21) Appl. No.: 17/259,969

(22) PCT Filed: Jul. 12, 2019
(86) PCT No.: PCT/US2019/041559

§ 371 Date: Jan. 13, 2021

Related U.S. Application Data
(60) Provisional application No. 62/697,746, filed on Jul. 13, 2018.

(51) Int. Cl. C07K 16/28; G01N 33/574; A61P 35/00

(52) U.S. Cl. CPC C07K 16/2878; C07K 16/2818; A61K 2039/507; G01N 33/57484; A61P 35/00; C07K 16/2827

(57) ABSTRACT

Provided are methods for clinical treatment of malignant tumors (e.g., advanced solid tumors) using a combination of an OX40 agonist, a PD-1 pathway inhibitor, and a CTLA-4 inhibitor.

### CROSS-REFERENCE TO RELATED APPLICATIONS

This PCT application claims the priority benefit of U.S. Provisional Application No. 62/697,746, filed Jul. 13, 2018, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 3338_125PC01_SeqListing_ST25.txt; Size: 133,135 bytes; and Date of Creation: Jul. 11, 2019) filed with the application is herein incorporated by reference in its entirety.

### FIELD OF THE DISCLOSURE

The present disclosure provides methods for treating a malignant tumor (e.g., advanced solid tumors) with a pharmaceutical composition comprising a combination of an OX40 agonist, a PD-1 pathway inhibitor, and a CTLA-4 inhibitor.

### BACKGROUND OF THE DISCLOSURE

Cancer immunotherapy has become well-established in recent years and is now one of the more successful treatment options available for patients with hematological malignancies and solid tumors. Scott, A. M., et al., Cancer Immun 12:14 (2012). Aside from targeting antigens that are involved in cancer cell proliferation and survival, antibodies can also activate or antagonize immunological pathways that are important in cancer immune surveillance. And, intensive efforts have led to the successful development of several immune checkpoint pathway inhibitors, some of which have been approved by the Food and Drug Administration, e.g., ipilimumab (YERVOY®), which binds to and inhibits CTLA-4 signaling (Hodi et al., N Engl J Med 363:711-723 (2010)); and nivolumab (OPDIVO®) and pembrolizumab (KEYTRUDA®), which binds to and inhibits the PD-1 signaling pathway (Topalian et al., N Engl J Med 366:2443-54 (2012a); Topalian et al., Curr Opin Immunol 24:207-12 (2012b); Topalian et al., J Clin Oncol 32(10):1020-30 (2014); Hamid et al., N Engl J Med 369:134-144 (2013); Hamid and Carvajal, Expert Opin Biol Ther 13(6):847-61 (2013); and McDermott and Atkins, Cancer Med 2(5):662-73 (2013)).

Despite such advances, patients with certain malignant tumors (e.g., metastatic or refractory solid tumors) continue to have very poor prognosis (Rosenberg S A, et al., Cancer immunotherapy in Cancer: Principles & Practice of Oncology (Eds DeVita V T, Lawrence T S and Rosenberg S A) 2011; 332-344 (Lippincott Williams & Wilkins, Philadelphia Pa.)). Only a subset of such patients actually experience long-term cancer remission, with many patients either not responding or initially responding but eventually developing resistance to the antibodies. Sharma, P., et al., Cell 168(4): 707-723 (2017). Accordingly, there has been growing interest in the use of these immune checkpoint inhibitors to target multiple non-redundant molecular pathways. However, not all combinations have acceptable safety and/or efficacy. Moreover, administering multiple antibodies can be burdensome due to different dosing and dosing intervals, which can require multiple injections at different time points. Therefore, there remains a need for a new treatment option with an acceptable profile and high efficacy that enhances antitumor immune responses compared to monotherapy and other immunotherapy combinations, as well as new pharmacodynamics markers to monitor efficacy of the treatment.

### SUMMARY OF THE DISCLOSURE

Provided herein is a method of treating a cancer or a solid tumor in a subject in need thereof comprising administering a therapeutically effective amount of: (a) an OX40 agonist; (b) a PD-1 pathway inhibitor; and (c) a CTLA-4 inhibitor to said subject.

In some embodiments, administering the composition disclosed herein (e.g., an

OX40 agonist, a PD-1 pathway inhibitor, and a CTLA-4 inhibitor in combination) inhibits and/or reduces a rate of tumor growth in the subject.

In some embodiments, the tumor volume is decreased by at least 5%, at least 10%, at least 20%, at least 30%, at least 40%, at least 50%, at least 60%, at least 70%, at least 80%, at least 90%, or 100% compared to a reference (e.g., corresponding tumor volume in a subject who did not receive a therapeutically effective amount of the OX40 agonist, the PD-1 pathway inhibitor, and the CTLA-4 inhibitor). In certain embodiments, the tumor volume is decreased by about 68% compared to a reference (e.g., corresponding tumor volume in a subject who received a dual therapy of anti-CTLA-4 antibody and anti-PD-1 antibody). In other embodiments, the tumor volume is decreased by about 73% compared to a reference (e.g., corresponding tumor volume in a subject who received a dual therapy of anti-CTLA-4 antibody and anti-PD-1 antibody). In further embodiments, the tumor volume is decreased by about 30% compared to a reference (e.g., corresponding tumor volume in a subject who received a dual therapy of anti-CTLA-4 antibody and anti-PD-1 antibody).

In some embodiments, the tumor weight is decreased by at least 5%, at least 10%, at least 20%, at least 30%, at least 40%, at least 50%, at least 60%, at least 70%, at least 80%, at least 90%, or at least 100% compared to a reference (e.g., corresponding tumor weight in a subject who did not receive a therapeutically effective amount of the OX40 agonist, the PD-1 pathway inhibitor, and the CTLA-4 inhibitor). In certain embodiments, the tumor weight is decreased by about 40% compared to a reference (e.g., corresponding tumor volume in a subject who received a dual therapy of anti-CTLA-4 antibody and anti-PD-1 antibody).

In some embodiments, the administering increases the frequency of effector CD4⁺ T cells in a tumor in the subject. In some embodiments, the administering increases the frequency of effector CD4⁺ T cells in a peripheral blood in the subject. In certain embodiments, the frequency of effector CD4⁺ T cells in the tumor and/or in the peripheral blood is increased by at least 5%, at least 10%, at least 20%, at least 30%, at least 40%, at least 50%, at least 60%, at least 70%, at least 80%, at least 90%, or at least 100% compared to a reference. In some embodiments, the frequency of effector CD4⁺ T cells in the tumor is increased by about 100% compared to a reference (e.g., corresponding value in a subject who received a dual therapy of anti-CTLA-4 antibody and anti-PD-1 antibody). In other embodiments, the frequency of effector CD4⁺ T cells in the peripheral blood is increased by about 70% compared to a reference (e.g., corresponding value in a subject who received a dual therapy of anti-CTLA-4 antibody and anti-PD-1 antibody). In some embodiments, the effector CD4⁺ T cells are Foxp3⁻ Ki67⁺.

In some embodiments, the administering increases the frequency of effector CD8⁺ T cells in peripheral blood in the subject. In certain embodiments, the frequency of effector CD8⁺ T cells in the peripheral blood is increased by at least 5%, at least 10%, at least 20%, at least 30%, at least 40%, at least 50%, at least 60%, at least 70%, at least 80%, at least 90%, or at least 100% compared to a reference. In certain embodiments, the frequency of effector CD8⁺ T cells in the peripheral blood is increased by about 33% compared to a reference (e.g., corresponding value in a subject who received a dual therapy of anti-CTLA-4 antibody and anti-PD-1 antibody). In some embodiments, the effector CD8⁺ T cells are PD1-Ki67⁺.

In some embodiments, the administering reduces the frequency of regulatory CD4⁺ T cells in a tumor in the subject. In certain embodiments, the frequency of regulatory CD4⁺ T cells in the tumor is reduced by at least 5%, at least 10%, at least 20%, at least 30%, at least 40%, at least 50%, at least 60%, at least 70%, at least 80%, at least 90%, or 100% compared to a reference. In some embodiments, the frequency of regulatory T cells in the tumor is reduced by about 30% compared to a reference (e.g., corresponding value in a subject who received a dual therapy of anti-CTLA-4 antibody and anti-PD-1 antibody). In certain embodiments, the regulatory CD4⁺ T cells are Foxp3⁺.

In some embodiments, the administering reduces the frequency of exhausted T cells in a tumor in the subject. In some embodiments, the frequency of exhausted T cells in the tumor is reduced by at least 5%, at least 10%, at least 20%, at least 30%, at least 40%, at least 50%, at least 60%, at least 70%, at least 80%, at least 90%, or 100% compared to a reference. In certain embodiments, the frequency of exhausted T cells in the tumor by about 71% compared to a reference (e.g., corresponding value in a subject who received a dual therapy of anti-CTLA-4 antibody and anti-PD-1 antibody).

In some embodiments, the administering reduces the frequency of granulocytic myeloid-derived suppressor cells (gMDSC) in a peripheral blood in the subject. In some embodiments, the frequency of gMDSC in the peripheral blood is reduced by at least 5%, at least 10%, at least 20%, at least 30%, at least 40%, at least 50%, at least 60%, at least 70%, at least 80%, at least 90%, or 100% compared to a reference. In certain embodiments, the frequency of gMDSC in the peripheral blood is reduced by about 30% compared to a reference (e.g., corresponding value in a subject who received a dual therapy of anti-CTLA-4 antibody and anti-PD-1 antibody).

In some embodiments, the administering increases a mean fluorescence intensity (MFI) of PD-L1 expression on a gMDSC in a tumor in the subject. In certain embodiments, the mean fluorescence intensity (MFI) of PD-L1 expression is increased by at least 5%, at least 10%, at least 20%, at least 30%, at least 40%, at least 50%, at least 60%, at least 70%, at least 80%, at least 90%, or at least 100% compared to a reference. In some embodiments, the MFI of PD-L1 expression is increased by about 89% compared to a reference (e.g., corresponding value in a subject who received a dual therapy of anti-CTLA-4 antibody and anti-PD-1 antibody).

In some embodiments, the administering reduces a mean fluorescence intensity

(MFI) of CCR2 expression on a monocyte in a peripheral blood in the subject. In certain embodiments, the mean fluorescence intensity (MFI) of CCR2 expression on the gMDSC is reduced increased by at least 5%, at least 10%, at least 20%, at least 30%, at least 40%, at least 50%, at least 60%, at least 70%, at least 80%, at least 90%, or at least 100% compared to a reference. In some embodiments, the MFI of CCR2 expression is reduced by about 54% compared to a reference (e.g., corresponding value in a subject who received a dual therapy of anti-CTLA-4 antibody and anti-PD-1 antibody). In some embodiments, the gMDSC is Ly6G⁺⁺.

In some embodiments, the administering increases a level of a proinflammatory cytokine in a tumor in the subject. In some embodiments, the level of the proinflammatory cytokine in the tumor is increased by at least 5%, at least 10%, at least 20%, at least 30%, at least 40%, at least 50%, at least 60%, at least 70%, at least 80%, at least 90%, or at least 100% compared to a reference. In certain embodiments, the proinflammatory cytokine comprises IFN-γ. In some embodiments, the level of IFN-γ is increased by about 39% compared to a reference (e.g., corresponding value in a subject who received a dual therapy of anti-CTLA-4 antibody and anti-PD-1 antibody).

In some embodiments, the administering increases the frequency of classical monocytes in the peripheral blood in the subject. In some embodiments, the frequency of classical monocytes is increased by at least 5%, at least 10%, at least 20%, at least 30%, at least 40%, at least 50%, at least 60%, at least 70%, at least 80%, at least 90%, or at least 100% compared to a reference (e.g., corresponding value in a subject who received a dual therapy of anti-CTLA-4 antibody and anti-PD-1 antibody). In certain embodiments, the frequency of classical monocytes is increased by about 80% compared to a reference (e.g., corresponding value in a subject who received a dual therapy of anti-CTLA-4 antibody and anti-PD-1 antibody). In some embodiments, the classical monocytes are Ly6C⁺⁺.

In some embodiments, the reference (e.g., against whom the above values are being compared) is a cancer subject who did not receive a therapeutically effective amount of the OX40 agonist, the CTLA-4 inhibitor, and the PD-1 inhibitor. In some embodiments, wherein the reference is a cancer subject who received an anti-PD-1 antibody monotherapy, an anti-CTLA-4 antibody monotherapy, or a dual combination of an anti-PD-1 antibody and an anti-CTLA-4 antibody.

In some embodiments, the OX40 agonist, the CTLA-4 inhibitor, and the PD-1 inhibitor are administered to the subject concurrently. In other embodiments, the OX40 agonist, the CTLA-4 inhibitor, and the PD-1 inhibitor are administered to the subject sequentially. In certain embodiments, the OX40 agonist, the CTLA-4 inhibitor, and the PD-1 inhibitor are administered to the subject in any order.

In some embodiments, the OX40 agonist is an anti-OX40 antibody. In some embodiments, the anti-OX40 antibody comprises a heavy chain CDR1, CDR2, and CDR3, and a light chain CDR1, CDR2, and CDR3, wherein (a) the heavy chain CDR1 comprises an amino acid sequence set forth as SEQ ID NO: 1, 9, 21, 29, 41, 49, 57, 65, or 77;

(b) the heavy chain CDR2 comprises an amino acid sequence set forth as SEQ ID NO: 2, 10, 22, 30, 42, 50, 58, 66, 78, or 85;

(c) the heavy chain CDR3 comprises an amino acid sequence set forth as SEQ ID NO: 3, 11, 23, 31, 43, 51, 59, 67, or 79;

(d) the light chain CDR1 comprises an amino acid sequence set forth as SEQ ID NO: 4, 12, 15, 24, 32, 35, 44, 52, 60, 68, 71, or 80;

(e) the light chain CDR2 comprises an amino acid sequence set forth as SEQ ID NO: 5, 13, 16, 25, 33, 36, 45, 53, 61, 69, 72, or 81; and

(f) the light chain CDR3 comprises an amino acid sequence set forth as SEQ ID NO: 6, 14, 17, 26, 34, 37, 46, 54, 62, 70, 73, or 82.

In some embodiments, the anti-OX40 antibody comprises a heavy chain variable region (VH) and a light chain variable region (VL), wherein the VH comprises an amino acid sequence set forth as SEQ ID NO: 7, 18, 27, 38, 47, 55, 63, 74, 83, or 86, and wherein the VL comprises an amino acid sequence set forth as SEQ ID NO: 8, 19, 20, 28, 39, 40, 48, 56, 64, 75, 76, or 84.

In some embodiments, the anti-OX40 antibody comprises a VH and a VL, wherein the VH comprises the CDR1, CDR2, and CDR3 sequences set forth as SEQ ID NOs: 77, 85, and 79, respectively, and wherein the VL comprises the CDR1, CDR2, and CDR3 sequences set forth as SEQ ID NOs: 80, 81, and 82, respectively.

In some embodiments, the anti-OX40 antibody is tavolixizumab (MEDI-0562), pogalizumab (MOXR0916, RG7888), GSK3174998, ATOR-1015, MEDI-6383, MEDI-6469, BMS986178, PF-04518600, RG7888 (MOXR0916), INCAGN1949, KHK4083, ENUM004, or ABBV-368.

In some embodiments, the CTLA-4 inhibitor is an anti-CTLA-4 antibody. In some embodiments, the anti-CTLA-4 antibody is ipilimumab (YERVOY®), tremelimumab (ticilimumab; CP-675,206), AGEN-1884, or ATOR-1015.

In some embodiments, the PD-1 inhibitor is an anti-PD-1 antibody. In certain embodiments, the anti-PD-1 antibody is nivolumab)(OPDIVO®), pembrolizumab (KEYTRUIDA®; MK-3475), pidilizumab (CT-011), PDR001, MEDI0680 (AMP-514), TSR-042, REGN2810, JS001, PF-06801591, BGB-A317, BI 754091, or SHR-1210.

In some embodiments, the PD-1 inhibitor is an anti-PD-L1 antibody. In certain embodiments, the anti-PD-L1 antibody is atezolizumab (TECENTRIQ®; RG7446; MPDL3280A; R05541267), durvalumab (MEDI4736), BMS-936559, avelumab (BAVENCIO®), LY3300054, CX-072 (Proclaim-CX-072), FAZ053, KN035, or MDX-1105.

In some embodiments, the PD-1 inhibitor is an anti-PD-L2 antibody. In certain embodiments, the anti-PD-L2 antibody is rHIgM12B7.

In some embodiments, the PD-1 inhibitor is a soluble protein. In other embodiments, an anti-PD-1 inhibitor is a fusion protein that specifically binds to PD-1. Non-limiting example of such a fusion protein is AMP-224 (GSK-2661380), which is composed of the extracellular domain of PD-L2 (B7-DC) fused to the Fc portion of IgG1.

In some embodiments, the cancer which the present methods can treat is selected from the group consisting of a liver cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, cancers of the childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combination thereof

In some embodiments, the cancer is melanoma (e.g., metastatic malignant melanoma), renal cancer (e.g. clear cell carcinoma), prostate cancer (e.g. hormone refractory prostate adenocarcinoma), breast cancer, colon cancer, lung cancer (e.g. non-small cell lung cancer), or any combination thereof. In certain embodiments, the cancer is breast cancer. In some embodiments, the breast cancer is triple negative breast cancer.

In some embodiments, the cancer that can be treated with the present methods is refractory to a first line treatment. In some embodiments, the cancer is refractory to a monotherapy comprising an OX40 inhibitor, a CTLA-4 inhibitor, or a PD-1 inhibitor. In some embodiments, the cancer is refractory to a doublet combination therapy comprising an OX40 inhibitor, a CTLA-4 inhibitor, or a PD-1 inhibitor.

In some embodiments, the above methods further comprise administering at least one additional therapeutic agent.

Also provided herein is a method of expanding effector CD4⁺ T cell populations in a human patient comprising administering a therapeutically effective amount of: (a) an OX40-agonist; (b) a PD-1 pathway inhibitor; and (c) a CTLA-4 inhibitor to said patient. In some embodiments, the effector CD4⁺ T cells are CD4⁺ Foxp3⁻. In certain embodiments, the expansion occurs in both malignant tumors and blood.

The present disclosure further provides a method of reducing exhausted effector CD4⁺ T cells in a human patient comprising administering a therapeutically effective amount of: (a) an OX40-agonist; (b) a PD-1 pathway inhibitor; and (c) a CTLA-4 inhibitor to said patient.

Also disclosed herein is a method of reducing the presence of granulocytic myeloid-derived suppressor cells in a human patient comprising administering a therapeutically effective amount of: (a) an OX40-agonist; (b) a PD-1 pathway inhibitor; and (c) a CTLA-4 inhibitor to said patient.

Provided herein is a method of increasing proinflammatory cytokine production by tumors in a human patient comprising administering a therapeutically effective amount of: (a) an OX40-agonist; (b) a PD-1 pathway inhibitor; and (c) a CTLA-4 inhibitor to said patient.

In some aspects, the present disclosure provides a method of treating a triple negative breast cancer in a subject in need thereof comprises administering to the subject a therapeutically effective amount of: (i) BMS986178, (ii) nivolumab (OPDIVO), and (iii) ipilimumab)(YERVOY®).

In some aspects, disclosed herein is a method of treating a triple negative breast cancer in a subject in need thereof comprises administering to the subject a therapeutically effective amount of: (i) tavolixizumab (MEDI-0562), (ii) durvalumab (IMFINZI®); MEDI4736), and (iii) tremelimumab (ticilimumab and CP-675,206).

Provided herein is a method of treating a triple negative breast cancer in a subject in need thereof comprises administering to the subject a therapeutically effective amount of: (i) pogalizumab (MOXR0916, RG7888), (ii) atezolizumab (TECENTRIQ®, RG7446), and (iii) ipilimumab (YERVOY®).

The present disclosure further provides a method of determining the efficacy of a cancer treatment in a subject, comprising measuring a frequency of effector CD4⁺ T cells (e.g., Foxp3⁻, Ki67⁺) in a peripheral blood of the subject, wherein an increase in the frequency of effector CD4⁺ T cells compared to a reference value (e.g., corresponding value in the subject prior to treatment) indicates that the cancer treatment is efficacious.

The present disclosure also provides a method of determining the efficacy of a cancer treatment in a subject, comprising measuring a frequency of effector CD8⁺ T cells (e.g., PD1⁻, Ki67⁺) in a peripheral blood of the subject, wherein an increase in the frequency of effector CD8⁺ T cells compared to a reference value (e.g., corresponding value in the subject prior to treatment) indicates that the cancer treatment is efficacious.

In some aspects, provided herein is a method of determining the efficacy of a cancer treatment in a subject, comprising measuring a frequency of regulatory T cells (e.g., Foxp3⁺ CD4⁺ T cells) in a peripheral blood of the subject, wherein an increase in the frequency of effector CD4⁺ T cells compared to a reference value (e.g., corresponding value in the subject prior to treatment) indicates that the cancer treatment is efficacious. In some embodiments, the cancer treatment referred to in the above methods comprises administering to the subject a therapeutically effective amount of : (a) an OX40 agonist; (b) a PD-1 pathway inhibitor; and (c) a CTLA-4 inhibitor to the subject.

### BRIEF DESCRIPTION OF THE DRAWINGS/FIGURES

FIGS. 1A and 1B shows a comparison of the anti-tumor effect of the different treatment groups. FIG. 1A provides the mean tumor volume (mm³) at days 5, 7, and 11 post-tumor implantation. The different treatment groups included: (i) IgG control antibody (black circle), (ii) anti-PD-1 antibody alone (black square), (iii) anti-CTLA-4 antibody alone (black triangle), (iv) anti-PD-1 antibody+anti-CTLA-4 antibody (black diamond), (v) anti-OX40 antibody alone (open circle), (vi) anti-OX40 antibody+anti-PD-1 antibody (open square), (vii) anti-OX40 antibody+anti-CTLA4 antibody (open triangle), and (viii) anti-OX40 antibody+anti-PD-1 antibody+anti-CTLA-4 antibody (open diamond). The mean tumor volume is shown as mean±SEM. FIG. 1B provides the tumor weight (mg) at day 12 post-tumor implantation. The treatment groups are the same as those described in FIG. 1A. Tumor weight is shown both individually and as mean±SEM. In both FIGS. 1A and 1B, the p value between the IgG control group and the group receiving all three antibodies was calculated by two-way ANOVA. “*” indicates p<0.01.

FIGS. 2A-2F show a comparison of the frequency of different T cell populations in the tumor and peripheral blood of mice from the various treatment groups at day 12 post-tumor implantation. FIGS. 2A, 2B, and 2C provide the frequency of CD3⁺ T cells, CD4⁺ T cells, and CD8⁺ T cells, respectively, in the tumor. FIGS. 2D, 2E, and 2F provide the frequency of CD3⁺ T cells, CD4⁺ T cells, and CD8⁺ T cells, respectively, in the peripheral blood. In FIGS. 2A-2F, T cell frequency is shown as a percentage of total CD45⁺ cells, both individually and as mean±SEM. Treatment groups are the same as those described in FIGS. 1A and 1B and indicated along the x-axis.

FIGS. 3A-3D show a comparison of the frequency of regulatory and effector T cells in the tumor and peripheral blood of mice from the various treatment groups at day 12 post-tumor implantation. FIGS. 3A and 3B provide the frequency of CD4⁺ Foxp3⁺ Tregs and CD4⁺ Ki67⁺ effector T cells, respectively, in the tumor. FIGS. 3C and 3D provide the frequency of CD4⁺ Foxp3⁺ Tregs and CD4⁺ Ki67⁺ effector T cells, respectively, in the peripheral blood. Frequency is shown as a percentage of total CD4⁺ T cells in the tumor or in the peripheral blood, both individually and as mean±SEM. Treatment groups are the same as those described in FIGS. 1A and 1B and indicated along the x-axis.

FIGS. 4A-4H show a comparison of the frequency of different CD8⁺ T cell populations in the tumor and peripheral blood of mice from the various treatment groups at day 12 post-tumor implantation. FIG. 4A provides the frequency of PD1⁺ and Ki67⁻ CD8⁺ T cells in the tumor. FIG. 4B provides the frequency of PD1⁺ and Ki67⁺ CD8⁺ T cells in the tumor. FIG. 4C provides the frequency of PD1 and Ki67⁺ CD8⁺ T cells in the tumor. FIG. 4D provides the frequency of PD1- and Ki67⁻ T CD8⁺ T cells in the tumor. FIG. 4E provides the frequency of PD1⁺ and Ki67⁻ CD8⁺ T cells in the peripheral blood. FIG. 4F provides the frequency of PD1⁺ and Ki67⁺ CD8⁺ T cells in the peripheral blood. FIG. 4G provides the frequency of PD1⁻ and Ki67⁺ CD8⁺ T cells in the peripheral blood. FIG. 4H provides the frequency of PD1⁻ and Ki67⁻ CD8⁺ T cells in the peripheral blood. Frequency is shown as a percentage of total CD8⁺ T cells in the tumor or in the peripheral blood, both individually and as mean±SEM. Treatment groups are the same as those described in FIGS. 1A and 1B and indicated along the x-axis.

FIGS. 5A-5D provide a comparison of the different myeloid populations in the tumor of mice from the various treatment groups at day 12 post-tumor implantation. FIGS. 5A, 5B, and 5C show the frequency of granulocytic myeloid-derived suppressor cells (Ly6G⁺⁺), monocytes (Ly6C⁺⁺), and macrophages (F4/80⁺). The frequency is shown as a percentage of total CD45⁺ T cells in the tumor. FIG. 5D shows the mean fluorescence intensity of PD-L1 expression on the granulocytic myeloid-derived suppressor cells (Ly6G⁺⁺). In FIGS. 5A-5D, data are shown both individually and as mean±SEM. Treatment groups are the same as those described in FIGS. 1A and 1B and indicated along the x-axis.

FIGS. 6A-6D provide a comparison of the different myeloid populations in the peripheral blood of mice from the various treatment groups at day 12 post-tumor implantation. FIGS. 6A, 6B, and 6C show the frequency of granulocytic myeloid-derived suppressor cells (Ly6G⁺⁺), non-classical monocytes (Ly6C⁻ CD43⁺), and classical monocytes (Ly6C⁺⁺). The frequency is shown as a percentage of total CD45⁺ T cells in the peripheral blood. FIG. 6D shows the mean fluorescence intensity of CCR2 expression on the classical monocytes (Ly6C⁺⁺). In FIGS. 6A-6D, data are shown both individually and as mean±SEM. Treatment groups are the same as those described in FIGS. 1A and 1B and indicated along the x-axis.

FIGS. 7A and 7B provide a comparison of the IFN-γ (FIG. 7A) and CXCL1 (FIG.

7B) levels in the supernatant of tumors from mice of the different treatment groups. The treatment groups are the same as those described in FIGS. 1A and 1B and indicated along the x-axis. Each circle represents a tumor from a single mouse. Data are also shown as mean±SEM.

FIGS. 8A-8D provide a comparison of the tumor growth curve in mice from the different treatment groups. FIG. 8A shows the tumor growth curve for mice that received one of the following: isotype control antibody alone, anti-PD-1 antibody alone, anti-CTLA-4 antibody alone, or anti-PD-1 antibody+anti-CTLA-4 antibody. FIGS. 8B, 8C, and 8D show the tumor growth curve for mice that received the anti-OX40 antibody at one of three doses, 10 mg/kg (FIG. 8B), 1 mg/kg (FIG. 8C), or 0.1 mg/kg (FIG. 8D), alone or with anti-PD-1 antibody and/or anti-CTLA-4 antibody. Each line represents an individual mouse.

FIG. 9 provides a comparison of the mean body weight of mice from the different treatment groups during the dosing period (days 6-14 post-tumor implantation). The treatment groups are the same as in FIGS. 8A-8D.

### DETAILED DESCRIPTION OF THE DISCLOSURE

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “administering” refers to the physical introduction of a therapeutic agent (e.g., combination of an anti-OX40 antibody, an anti-PD-1 antibody, and an anti-CTLA-4 antibody) to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE), as used herein, is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region. The heavy chain constant region comprises three constant domains, Cₘ, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. A heavy chain may have the C-terminal lysine or not. Unless specified otherwise herein, the amino acids in the variable regions are numbered using the Kabat numbering system and those in the constant regions are numbered using the EU system. In one embodiment, an antibody is an intact antibody.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” includes monospecific, bispecific, or multi-specific antibodies, as well as a single chain antibody. In embodiments, the antibody is a bispecific antibody. In other embodiments, the antibody is a monospecific antibody. In some embodiments, one or more of the OX40 agonist, PD-1 pathway inhibitor, or CTLA-4 inhibitor are comprised in a monospecific, bispecific, or multi-specific antibody (e.g., an anti-OX40/anti-PD-1 bispecific antibody and an anti-CTLA-4 monospecific antibody; an anti-OX40/anti-CTLA-4 bispecific antibody and an anti-PD-1 monospecific antibody; an anti-OX40/anti-PD-L1 bispecific antibody and an anti-CTLA-4 monospecific antibody; an anti-CTLA-⁴/anti-PD-1 bispecific antibody and an anti-OX40 monospecific antibody; an anti-OX40/anti-CTLA-4 bispecific antibody and an anti-PD-L1 monospecific antibody; an anti-OX40/anti-PD-¹/anti-CTLA-4 trispecific antibody; or an anti-OX40/anti-PD-L1/anti-CTLA-4 trispecific antibody. As used herein, an “IgG antibody” has the structure of a naturally occurring IgG antibody, i.e., it has the same number of heavy and light chains and disulfide bonds as a naturally occurring IgG antibody of the same subclass. For example, an anti-OX40 IgG1, IgG2, IgG3 or IgG4 antibody consists of two heavy chains (HCs) and two light chains (LCs), wherein the two heavy chains and light chains are linked by the same number and location of disulfide bridges that occur in naturally occurring IgG1, IgG2, IgG3 and IgG4 antibodies, respectively (unless the antibody has been mutated to modify the disulfide bonds).

An “isolated antibody” refers to an antibody that is substantially free of other antibodies having different antigenic specificities (e.g., an isolated antibody that binds specifically to OX40 is substantially free of antibodies that bind specifically to antigens other than OX40). An isolated antibody that binds specifically to OX40 may, however, have cross-reactivity to other antigens, such as OX40 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-OX40 antibody binds specifically to OX40, an anti-PD-1 antibody binds specifically to PD-1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-OX40 antibody described herein, include:

(1) a Fab fragment (fragment from papain cleavage) or a similar monovalent fragment consisting of the VL, VH, LC and CH1 domains;

(2) a F(ab′)2 fragment (fragment from pepsin cleavage) or a similar bivalent fragment comprising two Fab fragments linked by a disulfide bridge at the hinge region;

(3) a Fd fragment consisting of the VH and CH1 domains;

(4) a Fv fragment consisting of the VL and VH domains of a single arm of an antibody,

(5) a single domain antibody (dAb) fragment (Ward et al., (1989) Nature 341:544-46), which consists of a VH domain;

(6) a bi-single domain antibody which consists of two Vii domains linked by a hinge (dual-affinity re-targeting antibodies (DARTS));

(7) a dual variable domain immunoglobulin; (8) an isolated complementarity determining region (CDR); and (9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a melanoma refers to a tumor that is the result of a metastasized melanoma. Because the distal tumor is derived from the pre-metastasis tumor, the “derived from” tumor can also comprise the pre-metastasis tumor, e.g., a tumor derived from a melanoma can comprise a melanoma.

As used herein, the term “OX40” refers to a receptor that is a member of the TNF-receptor superfamily, which binds to OX40 ligand (OX40-L). OX40 is also referred to as tumor necrosis factor receptor superfamily, member 4 (TNFRSF4), ACT35, IMD16, TXGP1L, and CD134. The term “OX40” as used herein includes human OX40 (hOX40), variants, isoforms, and species homologs of hOX40, and analogs having at least one common epitope with hOX40. The amino acid sequence of human OX40 precursor can be found under GeneBank Accession No. NP 003318.1.

As used herein, the term “Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

The term “Programmed Death Ligand-1” (PD-L1), as used herein, is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

As used herein, the term “Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

A “subject” includes any human or non-human animal. In some embodiments, the subject is a human. The terms “subject” and “patient” are used interchangeably herein. In certain embodiments, the subject is a human patient, e.g., in whom enhancement of an immune response would be desirable, e.g., a patient who is afflicted with a cancer.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment can refer to alleviation of at least one symptom of a solid tumor. Such effective treatment may, e.g., reduce patient pain, reduce the size and/or number of lesions, can reduce or prevent metastasis of a tumor, and/or can slow tumor growth.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, e.g., a component of a signaling pathway, that can be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In preferred embodiments, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

As used herein, the term “immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

“T effector” (“Teff”) cells refers to T cells (e.g., CD4⁺ and CD8⁺ T cells) with cytolytic activities as well as T helper (Th) cells, which secrete cytokines and activate and direct other immune cells, but does not include regulatory T cells (Treg cells). Compositions disclosed herein (e.g., combination of an anti-OX40 antibody, an anti-PD-1 antibody, and an anti-CTLA-4 antibody) activate and/or increase the frequency of Teff cells, e.g., CD4⁺ and CD8⁺ T cells, in a tumor or blood of a subject.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some embodiments, an “effective amount” is the amount of anti-OX40 antibody, the amount of anti-PD-1 antibody, and the amount of anti-CTLA-4 antibody, in combination, clinically proven to affect a significant decrease in cancer or slowing of progression of cancer, such as an advanced solid tumor.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, at least about 95%, or at least about 100% relative to untreated subjects.

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns.”

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

As used herein, “treatment” or “therapy” of a subject refers to any type of intervention or process performed on, or the administration of an active agent disclosed herein (e.g., an anti-OX40 antibody, an anti-PD-1 antibody, and an anti-CTLA-4 antibody) to the subject with the objective of reversing, alleviating, ameliorating, inhibiting, slowing down or preventing the onset, progression, development, severity or recurrence of a symptom, complication or condition, or biochemical indicia associated with a disease.

As used herein, a “body surface area (BSA)-based dose” or “weight-based dose” refers to a dose (e.g., of an anti-OX40 antibody, an anti-PD-1 antibody, and/or an anti-CTLA-4 antibody) that is adjusted to the body-surface area (BSA) of the individual patient. A BSA-based dose can be provided as mg/kg body weight. Various calculations have been published to arrive at the BSA without direct measurement, the most widely used of which is the Du Bois formula (see Du Bois D, Du Bois E F (June 1916) Archives of Internal Medicine 17 (6): 863-71; and Verbraecken, J. et al. (April 2006). Metabolism—Clinical and Experimental 55 (4): 515-24). Other exemplary BSA formulas include the Mosteller formula (Mosteller R D. N Engl J Med., 1987; 317:1098), the Haycock formula (Haycock G B, et al., J Pediatr 1978, 93:62-66), the Gehan and George formula (Gehan E A, George S L, Cancer Chemother Rep 1970, 54:225-235), the Boyd formula (Current, J D (1998), The Internet Journal of Anesthesiology 2 (2); and Boyd, Edith (1935), University of Minnesota. The Institute of Child Welfare, Monograph Series, No. x. London: Oxford University Press), the Fujimoto formula (Fujimoto S, et al., Nippon Eiseigaku Zasshi 1968; 5:443-50), the Takahira formula (Fujimoto S, et al., Nippon Eiseigaku Zasshi 1968; 5:443-50), and the Schlich formula (Schlich E, et al., Ernährungs Umschau 2010; 57:178-183).

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., an anti-OX40 antibody, an anti-PD-1 antibody, and/or an anti-CTLA-4 antibody). For example, a 60 kg person and a 100 kg person would receive the same dose of an antibody (e.g., 360 mg of an anti-PD-1 antibody).

The use of the term “fixed dose” with regard to a method of the disclosure means that two or more different anti-cancer agents in a single composition (e.g., an anti-OX40 antibody, an anti-PD-1 antibody, and an anti-CTLA-4 antibody) are present in the composition in particular (fixed) ratios with each other. In some embodiments, the fixed dose is based on the weight (e.g., mg) of the anti-cancer agents. In certain embodiments, the fixed dose is based on the concentration (e.g., mg/ml) of the anti-cancer agents.

As used herein, “subtherapeutic dose” means a dose of a therapeutic compound (e.g., an antibody and/or an agonist) that is lower than the usual or typical dose of the therapeutic compound when administered alone for the treatment of a hyperproliferative disease (e.g., cancer).

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

### II. Methods of the Disclosure

The present disclosure is directed to a method for treating a tumor or a subject afflicted with a tumor comprising administering to the subject a therapeutically effective amount of: (i) an OX40 agonist, (ii) a PD-1 pathway inhibitor, and (iii) a CTLA-4 inhibitor. In some embodiments, the OX40 agonist is an antibody, or an antigen-binding fragment thereof, that binds specifically to an OX40 and induces OX40 activity (“anti-OX40 antibody”). In some embodiments, the PD-1 pathway inhibitor is an antibody, or an antigen-binding fragment thereof, that binds specifically to a Programmed Death-1 (PD-1) and inhibits PD-1 activity (“anti-PD-1 antibody”). In certain embodiments, the PD-1 pathway inhibitor is an antibody, or an antigen-binding fragment thereof, that binds specifically to a Programmed Death Ligand 1 (PD-L1) and inhibits PD-L1 activity (“anti-PD-L1 antibody”). In some embodiments, the CTLA-4 inhibitor is an antibody, or an antigen-binding fragment thereof, that binds specifically to a CTLA-4 and inhibits CTLA-4 activity (“anti-CTLA-4 antibody).”

In some embodiments, administering a composition disclosed herein treats a tumor or a subject afflicted with a tumor by promoting and/or enhancing an immune response against a tumor antigen. In some embodiments, administering a composition of the present disclosure increases the frequency of effector T cells (e.g., CD4⁺ or CD8⁺ T cells) in a tumor of a subject. In some embodiments, administering the composition disclosed herein increases the frequency of effector T cells (e.g., CD4⁺ or CD8⁺ T cells) in a peripheral blood of a subject. In some embodiments, the frequency of effector T cells (e.g., CD4⁺ or CD8⁺ T cells) in the tumor is increased by at least 5%, at least 10%, at least 20%, at least 30%, at least 40%, at least 50%, at least 60%, at least 70%, at least 80%, at least 90%, or at least 100% compared to a reference (e.g., the corresponding frequency in a subject that did not receive the composition disclosed herein, e.g., a dual combination of anti-PD-1 and anti-CTLA-4 antibodies). In certain embodiments, the frequency of effector T cells (e.g., CD4⁺ or CD8⁺ T cells) in a tumor of the reference (e.g., a subject that did not receive the composition disclosed herein, e.g., a dual combination of anti-PD-1 and anti-CTLA-4 antibodies) is about 3% of the total CD45⁺ cells. In some embodiments, the frequency of effector T cells (e.g., CD4⁺ or CD8⁺ T cells) in the peripheral blood is increased by at least 5%, at least 10%, at least 20%, at least 30%, at least 40%, at least 50%, at least 60%, at least 70%, at least 80%, at least 90%, or at least 100% compared to a reference (e.g., the corresponding frequency in a subject that did not receive the composition disclosed herein, e.g., a dual combination of anti-PD-1 and anti-CTLA-4 antibodies). In certain embodiments, the frequency of effector CD4⁺ T cells in a peripheral blood of the reference (e.g., a subject that did not receive the composition disclosed herein, e.g., a dual combination of anti-PD-1 and anti-CTLA-4 antibodies) is about 10% of the total CD45⁺ cells. In certain embodiments, the frequency of effector CD8⁺ T cells in a peripheral blood of the reference (e.g., a subject that did not receive the composition disclosed herein, e.g., a dual combination of anti-PD-1 and anti-CTLA-4 antibodies) is about 3% of the total CD45⁺ cells.

In some embodiments, administering a composition disclosed herein reduces the frequency of regulatory T cells in a tumor of a subject. In some embodiments, the regulatory T cells are Foxp3⁺. In certain embodiments, the frequency of regulatory T cells in the tumor is decreased by at least 5%, at least 10%, at least 20%, at least 30%, at least 40%, at least 50%, at least 60%, at least 70%, at least 80%, at least 90%, or at least 100% compared to a reference (e.g., the corresponding frequency in a subject that did not receive the composition disclosed herein, e.g., a dual combination of anti-PD-1 and anti-CTLA-4 antibodies). In certain embodiments, the frequency of regulatory T cells in a tumor of the reference (e.g., a subject that did not receive the composition disclosed herein, e.g., a dual combination of anti-PD-1 and anti-CTLA-4 antibodies) is about 30% of total CD4⁺ T cells.

In some embodiments, administering the composition disclosed herein reduces the frequency of exhausted T cells (i.e., PD-1⁺, Ki67⁻, CD8⁺) in a tumor of a subject. In some embodiments, the frequency of exhausted T cells in the tumor is reduced by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the frequency of exhausted T cells in a tumor of the reference (e.g., a subject that did not receive the composition disclosed herein, e.g., a dual combination of anti-PD-1 and anti-CTLA-4 antibodies) is about 7% of total CD8⁺ T cells.

In some embodiments, administering a composition of the present disclosure reduces the frequency of granulocytic myeloid-derived suppressor cells (gMDSCs) (Ly6G⁺⁺) in a blood of a subject. In some embodiments, administering a composition disclosed herein reduces the frequency of non-classical monocytes (Ly6C⁻ CD43⁺) in a blood of a subject. In certain embodiments, the frequency of the gMDSCs and/or the frequency of the non-classical monocytes is reduced by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the frequency of gMDSCs in a peripheral blood of the reference (e.g., a subject that did not receive the composition disclosed herein, e.g., a dual combination of anti-PD-1 and anti-CTLA-4 antibodies) is about 60% of the total CD45⁺ cells. In certain embodiments, the frequency of non-classical monocytes in a peripheral blood of the reference (e.g., a subject that did not receive the composition disclosed herein, e.g., a dual combination of anti-PD-1 and anti-CTLA-4 antibodies) is about 0.7% of the total CD45⁺ cells.

In some embodiments, administering a composition disclosed herein increases a PD-L1 expression on tumor infiltrating gMDSCs. In some embodiments, the PD-L1 expression (e.g., as measured by mean fluorescence intensity (MFI)) is increased by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies), e.g., as determined by flow cytometry. In certain embodiments, the MFI of PD-L1 expression on tumor infiltrating gMDSCs in the reference (e.g., a subject that did not receive the composition disclosed herein, e.g., a dual combination of anti-PD-1 and anti-CTLA-4 antibodies) is about 190.

In some embodiments, administering a composition disclosed herein decreases a CCR2 expression on gMDSCs in a peripheral blood of a subject. In some embodiments, the CCR2 expression (e.g., as measured by mean fluorescence intensity (MFI)) is decreased by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies), e.g., as determined by flow cytometry. In certain embodiments, the MFI of CCR2 expression on tumor infiltrating gMDSCs in the reference (e.g., a subject that did not receive the composition disclosed herein, e.g., a dual combination of anti-PD-1 and anti-CTLA-4 antibodies) is about 480.

In some embodiments, administering a composition disclosed herein increases the level of IFN-γ in a tumor of a subject. In some embodiments, the IFN-γ level is increased by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the IFN-γ level in a tumor of the reference (e.g., a subject that did not receive the composition disclosed herein, e.g., a dual combination of anti-PD-1 and anti-CTLA-4 antibodies) is about 4601±1333 pg/mL/g.

In some embodiments, administering a composition disclosed herein decreases the level of CXCL1 in a tumor of a subject. In some embodiments, the CXCL1 level is decreased by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the CXCL1 level in a tumor of the reference (e.g., a subject that did not receive the composition disclosed herein, e.g., a dual combination of anti-PD-1 and anti-CTLA-4 antibodies) is about 20137±727 pg/mL/g.

In some embodiments, administering a composition disclosed herein inhibits and/or reduces tumor growth in a subject. In some embodiments, the tumor growth (e.g., tumor volume or weight) is reduced by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the tumor volume in the reference (e.g., subject that did not receive the composition disclosed herein, e.g., a dual combination of anti-PD-1 and anti-CTLA-4 antibodies) is about 954.6 mm³. In certain embodiments, the tumor weight in the reference (e.g., subject that did not receive the composition disclosed herein, e.g., a dual combination of anti-PD-1 and anti-CTLA-4 antibodies) is about 150 mg. In some embodiments, a median tumor growth inhibition (TGI) is increased by at least 5%, at least 10%, at least 20%, at least 30%, at least 40%, at least 50%, at least 60%, at least 70%, at least 80%, at least 90%, or 100% compared to a reference (e.g., the corresponding frequency in a subject that did not receive the composition disclosed herein, e.g., a dual combination of anti-PD-1 and anti-CTLA-4 antibodies). In certain embodiments, the median tumor growth inhibition (TGI) in the reference (e.g., subject that did not receive the composition disclosed herein, e.g., a dual combination of anti-PD-1 and anti-CTLA-4 antibodies) is about 9.8%. In some embodiments, the a median survival is increased by at least 1 day, 2 days, 3 days, 4 days, 5 days, 6 days or more compared to a reference (e.g., the corresponding frequency in a subject that did not receive the composition disclosed herein, e.g., a dual combination of anti-PD-1 and anti-CTLA-4 antibodies). In certain embodiments, the median survival in the reference (e.g., subject that did not receive the composition disclosed herein, e.g., a dual combination of anti-PD-1 and anti-CTLA-4 antibodies) is about 26 days.

In some embodiments, the term “reference,” as used herein, refers to a corresponding subject (e.g., a cancer subject) who did not receive a composition disclosed herein, e.g., a subject who received a dual combination of anti-PD-1 and anti-CTLA-4 antibodies. The term “reference” can also refer to a same cancer subject but prior to the administration of a composition disclosed herein. In certain embodiments, the term “reference” refers to an average of a population of subjects (e.g., cancer subjects).

In some embodiments, tumors that can be treated with the methods disclosed herein are derived from cancers that are typically responsive to immunotherapy and those that are not typically responsive to immunotherapy. In some embodiments, the cancers are cancers with solid tumors or blood malignancies (liquid tumors). Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some embodiments, a method of treating cancer in a subject in need thereof comprises administering to the subject a therapeutically effective amount of: (i) BMS986178, (ii) nivolumab (OPDIVO®), and (iii) ipilimumab)(YERVOY®). In further embodiments, a method of treating cancer in a subject in need thereof comprises administering to the subject a therapeutically effective amount of: (i) tavolixizumab (MEDI-0562), (ii) durvalumab (IMIFINZI®; MEDI4736), and (iii) tremelimumab (ticilimumab and CP-675,206). In other embodiments, a method of treating cancer in a subject in need thereof comprises administering to the subject a therapeutically effective amount of: (i) pogalizumab (MOXR0916, RG7888), (ii) atezolizumab (TECENTRIQ®, RG7446), and (iii) ipilimumab (YERVOY®).

In some embodiments, the tumor is derived from a melanoma, a renal cell carcinoma (RCC), a non-small cell lung carcinoma (NSCLC), a urothelial cancer (UC), a breast cancer, or any combination thereof. In certain embodiments, a method of treating melanoma, RCC, NSCLC, UC or breast cancer in a subject in need thereof comprises administering to the subject a therapeutically effective amount of: (i) BMS986178, (ii) nivolumab (OPDIVO®), and (iii) ipilimumab (YERVOY®). In further embodiments, a method of treating melanoma, RCC, NSCLC, UC or breast cancer in a subject in need thereof comprises administering to the subject a therapeutically effective amount of: (i) tavolixizumab (MEDI-0562), (ii) durvalumab (IMFINZI®; MEDI4736), and (iii) tremelimumab (ticilimumab and CP-675,206). In other embodiments, a method of treating melanoma, RCC, NSCLC, UC or breast cancer in a subject in need thereof comprises administering to the subject a therapeutically effective amount of: (i) pogalizumab (MOXR0916, RG7888), (ii) atezolizumab (TECENTRIQ®, RG7446), and (iii) ipilimumab (YERVOY®).

In some embodiments, the tumor is derived from breast cancer. In certain embodiments, the breast cancer is a triple negative breast cancer (TNBC). Accordingly, in some embodiments, a method of treating a triple negative breast cancer in a subject in need thereof comprises administering to the subject a therapeutically effective amount of: (i) an OX40 agonist (e.g., an anti-OX40 antibody, e.g., those disclosed herein), (ii) a PD-1 pathway inhibitor (e.g., an anti-PD-1 antibody, e.g., those disclosed herein), and (iii) a CTLA-4 inhibitor (e.g., an anti-CTLA-4 antibody, e.g., those disclosed herein). In certain embodiments, a method of treating a triple negative breast cancer in a subject in need thereof comprises administering to the subject a therapeutically effective amount of: (i) BMS986178, (ii) nivolumab (OPDIVO®), and (iii) ipilimumab (YERVOY®). In further embodiments, a method of treating a triple negative breast cancer in a subject in need thereof comprises administering to the subject a therapeutically effective amount of: (i) tavolixizumab (MEDI-0562), (ii) durvalumab (IMFINZI®; MEDI4736), and (iii) tremelimumab (ticilimumab and CP-675,206). In other embodiments, a method of treating a triple negative breast cancer in a subject in need thereof comprises administering to the subject a therapeutically effective amount of: (i) pogalizumab (MOXR0916, RG7888), (ii) atezolizumab (TECENTRIQ®, RG7446), and (iii) ipilimumab (YERVOY®).

In some embodiments, the methods described herein can also be used for treatment of metastatic cancers, unresectable, refractory cancers (e.g., cancers refractory to previous immunotherapy, e.g., with a blocking CTLA-4 or PD-1 antibody, alone or in combination), and/or recurrent cancers.

In some embodiments, a subject to be treated with the methods disclosed herein has received one, two, three, four, five or more prior cancer treatments. In other embodiments, the subject is treatment-naïve (i.e., has never received a prior cancer treatment). In some embodiments, the subject has progressed on other cancer treatments. In certain embodiments, the prior cancer treatment comprised an immunotherapy (e.g., with an anti-CTLA-4 antibody and/or an anti-PD-1 antibody). In other embodiments, the prior cancer treatment comprised a chemotherapy. In some embodiments, the tumor has reoccurred. In some embodiments, the tumor is metastatic. In other embodiments, the tumor is not metastatic.

In some embodiments, the subject has received a prior therapy to treat the tumor and the tumor has relapsed or is refractory. In some embodiments, the subject has received a prior immuno-oncology (I-O) therapy to treat the tumor and the tumor has relapsed or is refractory. In some embodiments, the subject has received more than one prior therapy to treat the tumor and the tumor has relapsed or is refractory. In certain embodiments, the subject has previously received either an anti-PD-1 or anti-PD-L1 antibody monotherapy, or an anti-CTLA-4 antibody monotherapy. In some embodiments, the subject has previously received a dual combination of an anti-PD-1 antibody and an anti-CTLA-4 antibody.

In some embodiments, the previous line of therapy comprises a chemotherapy. In some embodiments, the chemotherapy comprises a platinum-based therapy. In some embodiments, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain embodiments, the platinum-based therapy comprises cisplatin. In one particular embodiment, the platinum-based therapy comprises carboplatin.

In some embodiments, the therapy of the present disclosure (e.g., administration of an anti-OX40 antibody, an anti-PD-1 antibody, and an anti-CTLA-4 antibody, in combination) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to another subject who was not treated with the combination therapy disclosed herein (e.g., received only a monotherapy or a dual combination therapy, e.g., an anti-PD-1 antibody in combination with an anti-CTLA-4 antibody). In still other embodiments, the combination therapy disclosed herein increases the duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than) the duration of survival of the subject using, e.g., a dual combination therapy of an anti-PD-1 antibody and an anti-CTLA-4 antibody.

In some embodiments, the combination therapy of the present disclosure effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to another subject who was not treated with the combination therapy disclosed herein (e.g., received only a monotherapy or a dual combination therapy, e.g., an anti-PD-1 antibody in combination with an anti-CTLA-4 antibody).

In some embodiments, the therapy of the present disclosure effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another subject who was not treated with the combination therapy disclosed herein (e.g., received only a monotherapy or a dual combination therapy, e.g., an anti-PD-1 antibody in combination with an anti-CTLA-4 antibody).

In some embodiments, one or more of the therapeutic effects observed (e.g., in the peripheral blood) with the combination therapy disclosed herein can be used as a marker for anti-tumor efficacy. For instance, it is disclosed herein that administration of the combination therapy disclosed herein to a cancer subject results in an increase in the frequency of proliferating effector T cells (e.g., Ki67⁺) in the peripheral blood of the subject. Accordingly, in some embodiments, an increase in the frequency of effector CD4⁺ T cells (e.g., Foxp3⁻, Ki67⁺) in the peripheral blood of a subject compared to a reference value (e.g., corresponding value in the subject prior to treatment) indicates that the treatment (e.g., combination therapy disclosed herein) is efficacious. In some embodiments, an increase in the frequency of effector CD8⁺ T cells (e.g., PDF, Ki67⁺) in the peripheral blood of a subject compared to a reference value (e.g., corresponding value in the subject prior to treatment) indicates that the treatment (e.g., combination therapy disclosed herein) is efficacious.

In certain embodiments, an increase in the frequency of regulatory T cells (e.g., Foxp3⁺ CD4⁺ T cells) in the peripheral blood of a subject compared to a reference value (e.g., corresponding value in the subject prior to treatment) can also indicate that the treatment (e.g., combination therapy disclosed herein) is efficacious. In some embodiments, a decrease in the frequency of granulocytic myeloid-derived suppressor cells (gMDSCs) (Ly6G⁺⁺) in a blood of a subject compared to a reference value (e.g., corresponding value in the subject prior to treatment) indicates that the treatment (e.g., combination therapy disclosed herein) is efficacious. In further embodiments, a decrease in the CCR2 expression on gMDSCs in a peripheral blood of a subject compared to a reference value (e.g., corresponding value in the subject prior to treatment) indicates that the treatment (e.g., combination therapy disclosed herein) is efficacious. In some embodiments, an increase in the frequency of classical monocytes (Ly6C⁺⁺) in a blood of a subject compared to a reference value (e.g., corresponding value in the subject prior to treatment) indicates that the treatment (e.g., combination therapy disclosed herein) is efficacious. As used herein, classical monocytes are CD14^{high}CD16⁻.

In the above embodiments, a treatment is efficacious, where the treatment reduces and/or prevents one or more symptoms associated with a cancer (e.g., reduces tumor volume).

IIIa. Anti-OX40 Antibodies Useful for the Disclosure

Anti-OX40 antibodies (or VH/VL domains derived therefrom) suitable for use in the methods disclosed herein are known in the art. For example, the anti-OX40 antibodies disclosed in U.S. Pat. No. 9,644,032 B2, which is hereby incorporated by reference (e.g., 3F4, 14B6-1, 14B6-2, 23H3, 6E1-1, 6E1-2, 18E9, 8B11, 20B3, 14A2-1, 14A2-2, and 20C1) can be used with the current methods. In some embodiments, the anti-OX40 antibodies that can be used with the current methods include PF-04518600, INCAGN1949, GSK3174998, KHK4083, MEDI0562, RG7888 (MOXR0916), MEDI6469, ENUM004, ABBV-368, and ATOR-1015 (bispecific). Other art recognized anti-OX40 antibodies that can be used include those described in International Publication No. WO 2015/153513 A1, WO 2015/153513 A1, WO 2015/153514 A1, WO 2014/148895 A1, WO 2013/038191 A2, WO 2016/057667 A1, WO 2009/079335 A1, WO 2016/179517 A1, WO 2017/096179 A1, WO 2017/096281 A1, WO 2017/096182 A1, WO 2017/134292 A1, WO 2018/031490 A1, WO 2018/089628 A1, and U.S. Pat. No. 9,006,399 B2, all of which are hereby incorporated by reference. Antibodies that compete with any of the above-referenced art-recognized antibodies for binding to OX40 can also be used.

In some embodiments, an anti-OX40 antibody that is useful for the present methods comprises a heavy chain CDR1, CDR2, and CDR3, and a light chain CDR1, CDR2, and CDR3, wherein

(a) the heavy chain CDR1 comprises an amino acid sequence set forth as SEQ ID NO: 1, 9, 21, 29, 41, 49, 57, 65, or 77;

(b) the heavy chain CDR2 comprises an amino acid sequence set forth as SEQ ID NO: 2, 10, 22, 30, 42, 50, 58, 66, 78, or 85;

(c) the heavy chain CDR3 comprises an amino acid sequence set forth as SEQ ID NO: 3, 11, 23, 31, 43, 51, 59, 67, or 79;

(d) the light chain CDR1 comprises an amino acid sequence set forth as SEQ ID NO: 4, 12, 15, 24, 32, 35, 44, 52, 60, 68, 71, or 80;

(e) the light chain CDR2 comprises an amino acid sequence set forth as SEQ ID NO: 5, 13, 16, 25, 33, 36, 45, 53, 61, 69, 72, or 81; and

(f) the light chain CDR3 comprises an amino acid sequence set forth as SEQ ID NO: 6, 14, 17, 26, 34, 37, 46, 54, 62, 70, 73, or 82.

In some embodiments, an anti-OX40 antibody comprises a heavy chain variable region (VH) and a light chain variable region (VL), wherein the VH comprises an amino acid sequence set forth as SEQ ID NO: 7, 18, 27, 38, 47, 55, 63, 74, 83, or 86, and wherein the VL comprises an amino acid sequence set forth as SEQ ID NO: 8, 19, 20, 28, 39, 40, 48, 56, 64, 75, 76, or 84. In certain embodiment, the antibody competes for binding with and/or binds to the same epitope on OX40 as the above-mentioned antibodies. In other embodiments, the antibody binds to all or a portion of an epitope of human OX40 comprising the amino acid sequence DVVSSKPCKPCTWCNLR (SEQ ID NO: 87) or DSYKPGVDCAPCPPGHFSPGDNQACKPWTNCTLAGK (SEQ ID NO: 88) of mature extracellular portion of human OX40 (SEQ ID NO: 93). In some embodiments, an anti-OX40 antibody described herein bind to all or a portion of the sequence SQNTVCRPCGPGFYNDVVSSKPCKPCTWCNLR (SEQ ID NO: 89). In other embodiments, an anti-OX40 antibodies described herein bind to all or a portion of the sequence PCKPCTWCNLR (SEQ ID NO: 90). In certain embodiments, an anti-OX40 antibodies that bind to all or a portion of the sequence DVVSSKPCKPCTWCNLR (SEQ ID NO: 87) further bind to all or a portion of the sequence QLCTATQDTVCR (SEQ ID NO: 91). In further embodiments, an anti-OX40 antibodies described herein bind to all or a portion of the sequence SQNTVCRPCGPGFYN (SEQ ID NO: 92).

In some embodiments, an anti-OX40 antibody useful for the present disclosure comprises a VH and a VL, wherein the VH has at least about 90% sequence identity (e.g., at least about 90%, at least about 95%, at least about 97%, or at least about 99%) with an amino acid sequence set forth as SEQ ID NO: 7, 18, 27, 38, 47, 55, 63, 74, 83, or 86, and wherein the VL has at least about 90% sequence identity (e.g., at least about 90%, at least about 95%, at least about 97%, or at least about 99%) with an amino acid sequence set forth as SEQ ID NO: 8, 19, 20, 28, 39, 40, 48, 56, 64, 75, 76, or 84.

In some embodiments, an anti-OX40 antibody that can be used with the current methods comprises a VH and a VL, wherein the VH comprises the CDR1, CDR2, and CDR3 sequences set forth as SEQ ID NOs: 77, 85, and 79, respectively, and wherein the VL comprises the CDR1, CDR2, and CDR3 sequences set forth as SEQ ID NOs: 80, 81, and 82, respectively. In some embodiments, the anti-OX40 antibody comprises a VH and a VL, wherein the VH comprises the amino acid sequence set forth as SEQ ID NO: 86, and wherein the VL comprises the amino acid sequence set forth as SEQ ID NO: 84.

In some embodiments, an anti-OX40 antibody comprises a heavy chain and a light chain, wherein:

(a) the heavy chain comprises an amino acid sequence that is at least 80%, 85%, 90%, 95%, 96%, 97%, 98% or 99% identical to an amino acid sequence selected from the group consisting of SEQ ID NOs: 94, 96, 98, 100, 102, 104, 106, 108, 110, 112, 114, 116, 118, 120, 122, 123 and 124, or comprises 1, 2, 3, 4, 5, 1-2, 1-3, 1-4, 1-5, 1-10, 1-15, 1-20, 1-25, or 1-50 amino acid changes (i.e., amino acid substitutions, additions or deletions) relative to an amino acid sequence selected from the group consisting of SEQ ID NOs: 94, 96, 98, 100, 102, 104, 106, 108, 110, 112, 114, 116, 118, 120, 122, 123 and 124; and/or

(b) the light chain comprises an amino acid sequence that is at least 80%, 85%, 90%, 95%, 96%, 97%, 98% or 99% identical to an amino acid sequence selected from the group consisting of SEQ ID NOs: 95, 97, 99, 101, 103, 105, 107, 109, 111, 113, 115, 117, 119, and 121, or comprises 1, 2, 3, 4, 5, 1-2, 1-3, 1-4, 1-5, 1-10, 1-15, 1-20, 1-25, or 1-50 amino acid changes (i.e., amino acid substitutions, additions or deletions) relative to an amino acid sequence selected from the group consisting of SEQ ID NOs: 95, 97, 99, 101, 103, 105, 107, 109, 111, 113, 115, 117, 119, and 121.

In some embodiments, an anti-OX40 antibody useful for the present methods comprise a heavy chain CDR1, CDR2, and/or CDR3, and/or a light chain CDR1, CDR2, and/or CDR3 that differs from the corresponding CDR of 3F4, 14B6-1, 14B6-2, 23H3, 6E1-1, 6E1-2, 18E9, 8B11, 20B3, 14A2-1, 14A2-2, and/or 20C1, in 1, 2, 3, 4, 5, 1-2, 1-3, 1-4, or 1-5 amino acid changes (i.e., amino acid substitutions, additions or deletions). In certain embodiments, the antibody comprises 1-5 amino acid changes in each of 1, 2, 3, 4, 5 or 6 of the CDRs relative to the corresponding sequence in 3F4, 14B6-1, 14B6-2, 23H3, 6E1-1, 6E1-2, 18E9, 8B11, 20B3, 14A2-1, 14A2-2, and/or 20C1. In certain embodiments, the antibody comprises at total of 1-5 amino acid changes across all CDRs relative to the CDRs in 3F4, 14B6-1, 14B6-2, 23H3, 6E1-1, 6E1-2, 18E9, 8B11, 20B3, 14A2-1, 14A2-2, and/or 20C1.

In some embodiments, an anti-OX40 antibody tavolixizumab (MEDI-0562), pogalizumab (MOXR0916, RG7888), GSK3174998, ATOR-1015, MEDI-6383, MEDI-6469, BMS986178, PF-04518600, or RG7888 (MOXR0916).

IIIb. Anti-PD-1 Antibodies Useful for the Disclosure

Antibodies (e.g., human antibodies) that bind specifically to PD-1 with high affinity have been disclosed in U.S. Pat. Nos. 8,008,449 and 8,779,105, each of which is hereby incorporated by reference. Other anti-PD-1 mAbs have been described in, for example, U.S. Pat. Nos. 6,808,710, 7,488,802, 8,168,757 and 8,354,509, and PCT Publication No. WO 2012/145493, each of which is hereby incorporated by reference. Each of the anti-PD-1 HuMAbs disclosed in U.S. Patent No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-y production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 antibodies useful for the present invention include mAbs that bind specifically to human PD-1 and exhibit at least one, preferably at least five, of the preceding characteristics.

In some embodiments, the anti-PD-1 antibody is nivolumab. Nivolumab (also known as “OPDIVO®”; 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56, each of which is hereby incorporated by reference). In some embodiments, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

Anti-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121168, the teachings of which are hereby incorporated by reference, can be used. Other known PD-1 antibodies include lambrolizumab (MK-3475) described in WO 2008/156712, and AMP-514 described in WO 2012/145493, the teachings of which are hereby incorporated by reference. Further known anti-PD-1 antibodies and other PD-1 inhibitors include those described in WO 2009/014708, WO 03/099196, WO 2009/114335 and WO 2011/161699, the teachings of which are hereby incorporated by reference. Another known anti-PD-1 antibody is pidilizumab (CT-011). Antibodies or antigen binding fragments thereof that compete with any of these antibodies or inhibitors for binding to PD-1 also can be used.

In some embodiments, the anti-PD-1 antibody or antigen binding fragment thereof cross-competes with pembrolizumab. In some embodiments, the anti-PD-1 antibody or antigen binding fragment thereof binds to the same epitope as pembrolizumab. In certain embodiments, the anti-PD-1 antibody or antigen binding fragment thereof has the same CDRs as pembrolizumab. In another embodiment, the anti-PD-1 antibody is pembrolizumab. Pembrolizumab (also known as “KEYTRUIDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also worldwideweb.cancer.gov/drugdictionary?cdrid=695789 (last accessed: May 25, 2017), each of which is hereby incorporated by reference. Pembrolizumab has been approved by the FDA for the treatment of relapsed or refractory melanoma.

In other embodiments, the anti-PD-1 antibody or antigen binding fragment thereof cross-competes with MEDI0608. In still other embodiments, the anti-PD-1 antibody or antigen binding fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089 or in worldwideweb.cancer.gov/drugdictionary?cdrid=756047 (last accessed May 25, 2017), each of which is hereby incorporated by reference.

In other embodiments, the anti-PD-1 antibody or antigen binding fragment thereof cross-competes with BGB-A317. In some embodiments, the anti-PD-1 antibody or antigen binding fragment thereof binds the same epitope as BGB-A317. In certain embodiments, the anti-PD-1 antibody or antigen binding fragment thereof has the same CDRs as BGB-A317. In certain embodiments, the anti-PD-1 antibody or antigen binding fragment thereof is BGB-A317, which is a humanized monoclonal antibody. BGB-A317 is described in U.S. Publ. No. 2015/0079109, which is hereby incorporated by reference.

In some embodiments, antibodies or antigen binding fragments thereof that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Anti-PD-1 antibodies or antigen binding fragments thereof suitable for use in the disclosed compositions are antibodies that bind to PD-1 with high specificity and affinity, block the binding of PD-L1 and or PD-L2, and inhibit the immunosuppressive effect of the PD-1 signaling pathway. In certain embodiments, the anti-PD-1 antibody, or antigen-binding portion thereof, cross-competes with nivolumab for binding to human PD-1. In other embodiments, the anti-PD-1 antibody, or antigen-binding portion thereof, is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody or antigen binding fragment thereof comprises a heavy chain constant region which is of a human IgG1 or IgG4 isotype. In certain other embodiments, the sequence of the IgG4 heavy chain constant region of the anti-PD-1 antibody or antigen binding fragment thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody, or antigen binding fragment thereof, is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab (KEYTRUDA®). In other embodiments, the anti-PD-1 antibody is chosen from the human antibodies 17D8, 2D3, 4H1, 4A11, 7D3 and 5F4 described in U.S. Pat. No. 8,008,449, which is hereby incorporated by reference. In still other embodiments, the anti-PD-1 antibody is MEDI0608 (formerly AMP-514) or Pidilizumab (CT-011).

In some embodiments, an anti-PD-1 fusion protein can be used, as a replacement or in combination with anti-PD-1 antibodies disclosed herein. Non-limiting example of such a fusion protein is AMP-224 (GSK-2661380), which is composed of the extracellular domain of PD-L2 (B7-DC) fused to the Fc portion of IgG1.

IIIc. Anti-PD-L1 Antibodies Useful for the Disclosure

Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Other examples of an anti-PD-L1 antibody include atezolizumab (TECENTRIQ®; RG7446), or durvalumab (IMFINZI®; MEDI4736). Antibodies or antigen binding fragments thereof that compete with any of these art-recognized antibodies or inhibitors for binding to PD-L1 also can be used.

In certain embodiments, the anti-PD-L1 antibody is BMS-936559 (formerly 12A4 or MDX-1105) (see, e.g., U.S. Pat. No. 7,943,743; WO 2013/173223, both of which are hereby incorporated by reference). In other embodiments, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149, both of which are hereby incorporated by reference), MEDI4736 (Khleif, 2013, In: Proceedings from the European Cancer Congress 2013; Sep. 27-Oct. 1, 2013; Amsterdam, The Netherlands. Abstract 802, which is hereby incorporated by reference), or MSB0010718C (also called avelumab or BAVENCIO®; see US 2014/0341917, which is hereby incorporated by reference). In certain embodiments, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

IIId. Anti-CTLA-4 Antibodies Useful for the Disclosure

An anti-CTLA-4 antibody used with the methods disclosed herein binds to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

HuMAbs that bind specifically to CTLA-4 with high affinity have been disclosed in U.S. Pat. Nos. 6,984,720 and 7,605,238, each of which is hereby incorporated by reference. Other anti- CTLA-4 mAbs have been described in, for example, U.S. Pat. Nos. 5,977,318, 6,051,227, 6,682,736, and 7,034,121, each of which is hereby incorporated by reference . The anti-CTLA-4 HuMAbs disclosed in U.S. Pat. Nos. 6,984,720 and 7,605,238, both of which are hereby incorporated by reference, have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ 5⁻¹; and (d) inhibits the binding of CTLA-4 to B7-1 (CD80) and B7-2 (CD86). Anti-CTLA-4 antibodies useful for the present invention include mAbs that bind specifically to human CTLA-4 and exhibit at least one, at least two, or at least three of the preceding characteristics. An exemplary clinical anti-CTLA-4 antibody is the human mAb 10D1 (now known as ipilimumab and marketed as YERVOY®) as disclosed in U.S. Pat. No. 6,984,720, which is hereby incorporated by reference.

An exemplary clinical anti-CTLA-4 antibody is the human mAb 10D1 (now known as ipilimumab and marketed as YERVOY®) as disclosed in U.S. Pat. No. 6,984,720, which is hereby incorporated by reference. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

Another anti-CTLA-4 antibody useful for the present methods is tremelimumab (also known as ticilimumab and CP-675,206). Tremelimumab is human IgG2 monoclonal anti-CTLA-4 antibody. Tremelimumab is described in WO/2012/122444, U.S. Publ. No. 2012/263677, and WO Publ. No. 2007/113648 A2, each of which is hereby incorporated by reference.

Anti-CTLA-4 antibodies useful for the disclosed composition also include isolated antibodies that bind specifically to human CTLA-4 and cross-compete for binding to human CTLA-4 with ipilimumab or tremelimumab or bind to the same epitope region of human CTLA-4 as ipilimumab or tremelimumab. In certain embodiments, the antibodies that cross-compete for binding to human CTLA-4 with, or bind to the same epitope region of human CTLA-4 as does ipilimumab or tremelimumab, are antibodies comprising a heavy chain of the human IgG1 isotype. For administration to human subjects, these cross-competing antibodies are chimeric antibodies, or humanized or human antibodies. Antigen-binding portions of the above antibodies, such as Fab, F(ab′)₂, Fd or Fv fragments, can also be used with the present methods.

### IV. Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions can be employed as carriers, particularly for injectable solutions (e.g., comprising an anti-OX40 antibody, an anti-PD-1 antibody, and an anti-CTLA-4 antibody). Liquid compositions for parenteral administration can be formulated for administration by injection or continuous infusion. Routes of administration by injection or infusion include intravenous, intraperitoneal, intramuscular, intrathecal and subcutaneous. In some embodiments, the composition comprising an anti-OX40 antibody, an anti-PD-1 antibody, and an anti-CTLA-4 antibody are administered intravenously (e.g., in separate formulations or together (in the same formulation or in separate formulations)).

### V. Patient Populations

Provided herein are methods for treating solid tumors or cancer (e.g., advanced refractory solid tumors) in human patients using an OX40 agonist (e.g., anti-OX40 antibody) in combination with a PD-1 pathway inhibitor and a CTLA-4 inhibitor.

Non-limiting examples of cancers or solid tumors that can be treated using the methods disclosed herein, include liver cancer, bone cancer, pancreatic cancer, skin cancer, cancer of the head or neck, oral cancer breast cancer, lung cancer—including small cell and non-small cell lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is also applicable to treatment of metastatic cancers.

In some embodiments, the subject suffers from a bladder cancer, breast cancer, cervical cancer, renal cell carcinoma, testicular cancer, colorectal cancer, lung cancer, head and neck cancer, ovarian cancer, or any combination thereof. In some embodiments, the subject suffers from a breast cancer. In certain embodiments, the breast cancer is a triple negative breast cancer (TNBC). As used herein, the term “triple negative breast cancer” refers to a breast cancer that is characterized by a lack of detectable expression of estrogen receptor (ER), progesterone receptor (PR), and human epidermal growth factor receptor type 2 (HER2 or ErbB2).

In some embodiments, the subject being treated with the methods described herein has an advanced solid tumor. For example, in certain embodiments, the subject has cervical cancer. In other embodiments, the subject has colorectal cancer. In some embodiments, the subject has bladder cancer (e.g., unresectable locally advanced or metastatic bladder cancer). In yet other embodiments, the subject has ovarian cancer (e.g., unresectable locally advanced or metastatic ovarian cancer).

In some embodiments, the subject being treated with the methods described herein has non-small cell lung cancer (NSCLC). In other embodiments, the subject has squamous cell carcinoma of the head and neck (SCCHN). In some embodiments, the subject has B-cell non-Hodgkin's lymphoma (B-NHL). In certain embodiments, the subject has myeloma. In some embodiments, the subject has melanoma. In further embodiments, the subject has diffuse large B-cell lymphoma (DLBCL).

In some embodiments, the subject suffers from a bladder cancer. In certain embodiments, the bladder cancer is a locally advanced bladder cancer. In other embodiments, the bladder cancer is a metastatic bladder cancer. In some embodiments, the bladder cancer is a metastatic urothelial bladder cancer. In some embodiments, the bladder cancer is advanced urothelial carcinoma. In certain embodiments, the subject suffers from a histologically or cytologically confirmed urothelial carcinoma (including mixed histologies of urothelial carcinoma with elements of other subtypes) of the renal pelvis, ureter, bladder, or urethra with progression or refractory disease. In some embodiments, the subject suffers from a bladder cancer and has been offered and/or have received or refused 1 prior platinum-based therapy for the treatment of metastatic or locally advanced unresectable disease. In certain embodiments, the subject has not received more than 1 prior systemic therapy. In some embodiments, the subject is immunotherapy treatment naïve (e.g., no prior therapy with experimental anti-tumor vaccines; any T-cell co-stimulation or checkpoint pathways, such as anti-PD-1, anti-PD-L1, anti-PD-L2, anti-CD137, or anti-CTLA-4 antibody, including ipilimumab; or other medicines specifically targeting T-cells). In some embodiments, the subject had received peri-operative (neo-adjuvant or adjuvant) treatment with a platinum agent. In some embodiments, the patient had received peri-operative (neo-adjuvant or adjuvant) treatment with a platinum agent in the setting of cystectomy for localized muscle invasive urothelial cancer. In certain embodiments, the patient had received peri-operative (neo-adjuvant or adjuvant) treatment with a platinum agent in the setting of cystectomy for localized muscle invasive urothelial cancer in the prior 12 months. Sequential chemotherapy given as a planned sequence to optimize response will count as 1 regimen.

In some embodiments, the human patient suffers from cervical cancer. In one embodiment, the cervical cancer is unresectable, metastatic, or recurrent with documented disease progression.

In some embodiments, the human patient suffers from renal cell carcinoma. In one embodiment, the renal cell carcinoma is metastatic renal cell carcinoma. In one embodiment, the renal cell carcinoma is a renal cell carcinoma with a clear-cell component.

In some embodiments, the human patient suffers from testicular cancer,.

In some embodiments, the human patient suffers from colorectal cancer. In certain embodiments, the colorectal cancer is a microsatellite instability-high (MSI-H) colorectal cancer. In other embodiments, the colorectal cancer is a microsatellite stable colorectal cancer. In further embodiments, the colorectal cancer is a mismatch repair-deficient colorectal cancer.

In some embodiments, the human patient suffers from lung cancer. In some embodiments, the human patient suffers from non-small cell lung cancer.

In some embodiments, the human patient suffers from head and neck cancer. In certain embodiments, the head and neck cancer is squamous cell carcinoma.

In some embodiments, the human patient suffers from ovarian cancer. In some embodiments, the ovarian cancer is unresectable locally advanced ovarian cancer. In certain embodiments, the ovarian cancer is metastatic ovarian cancer. In some embodiments, the ovarian cancer is recurrent platinum-sensitive ovarian cancer.

In some embodiments, the human patient suffers from melanoma.

In some embodiments, the human patient suffers from gastric adenocarcinoma

In some embodiments, the subject suffers from a non-small cell lung cancer (NSCLC) or a virally-related cancer (e.g., a human papilloma virus (HPV)-related tumor) or gastric adenocarcinoma. In certain embodiments, the HPV-related tumor is HPV+ head and neck cancer (HNC). In some embodiments, the gastric adenocarcinoma is associated with Epstein-Barr virus (EBV) infection.

In some embodiments, the methods described herein are used to treat patients having a cancer that exhibited an inadequate response to a prior treatment, e.g., a prior treatment with an immuno-oncology drug, or patients having a cancer that is refractory or resistant, either intrinsically refractory or resistant (e.g., refractory to a PD-1 pathway antagonist), or a wherein the resistance or refractory state is acquired. For example, subjects who are not responsive or not sufficiently responsive to a first therapy or who see disease progression following treatment, e.g., anti-PD-1 and/or an anti-CTLA-4 antibody treatment, can be treated by the methods described herein.

In some embodiments, the methods described herein are used to treat patients who have not previously received (i.e., been treated with) an immuno-oncology agent, e.g., a PD-1 pathway antagonist and/or a CTLA-4 inhibitor.

In some embodiments, the methods described herein are used in combination with a standard of care treatment (e.g., surgery, radiation, and chemotherapy). In other embodiments, the methods described herein are used as a maintenance therapy, e.g., a therapy that is intended to prevent the occurrence or recurrence of tumors.

In some embodiments, the methods described herein are used with another treatment, e.g., radiation, surgery, or chemotherapy. For example, the methods described herein can be used when there is a risk that micrometastases can be present and/or in order to reduce the risk of a relapse

Patients can be tested or selected for one or more of the above described clinical attributes prior to, during or after treatment.

### VI. Combination Therapy

Combination therapies provided herein involve administering an OX40 agonist, in combination with a PD-1 pathway inhibitor and a CTLA-4 inhibitor, to treat subjects having a cancer, e.g., a solid tumor (e.g., an advanced refractory solid tumor). In some embodiments, the present disclosure provides an OX40 agonist, a PD-1 pathway inhibitor, and a CTLA-4 inhibitor, in combination, according to a defined clinical dosage regimen, to treat subjects having a malignant tumor (e.g., an advanced refractory solid tumor). In some embodiments, the OX40 agonist is an anti-OX40 antibody, such as those disclosed herein. In some embodiments, the PD-1 pathway inhibitor is an anti-PD-1 antibody or an anti-PD-L1 antibody, such as those disclosed herein. In some embodiments, the CTLA-4 inhibitor is an anti-CTLA-4 antibody, such as those disclosed herein. In some embodiments, dosage regimens are adjusted to provide the optimum desired response (e.g., an effective response).

As used herein, adjunctive or combined administration (co-administration) includes simultaneous administration of the compounds in the same or different dosage form, or separate administration of the compounds (e.g., sequential administration). Thus, in some embodiments, the anti-OX40 antibody, the anti-PD-1 antibody, and the anti-CTLA-4 antibody are simultaneously administered in a single formulation. In other embodiments, the anti-OX40 antibody, the anti-PD-1 antibody, and the anti-CTLA-4 antibody are formulated for separate administration and are administered concurrently or sequentially (e.g., one antibody is administered within about 30 minutes (e.g., within about 29, 28, 27, 26, 25, 24, 23, 22, 21, 20, or less minutes) prior to administration of the second antibody), and in any order.

### VII. Treatment Protocols

Suitable treatment protocols for treating a malignant tumor in a human patient include, for example, administering to the patient an effective amount of each of: (i) an anti-OX40 antibody, (ii) an anti-PD-1 antibody, and (iii) an anti-CTLA-4 antibody, wherein the method comprises at least one administration cycle, wherein for each of the at least one cycle, (a) at least one dose of the anti-OX40 antibody is administered at a flat dose of about 1, 3, 10, 20, 40, 50, 80, 100, 130, 150, 160, 180, 200, 240, or 320 mg, (b) at least one dose of the anti-PD-1 antibody is administered at a flat dose of about 50, 80, 100, 120, 150, 180, 200, 240, 360, 480, 720, or 960 mg, and (c) at least one dose of the anti-CTLA-4 antibody is administered at a flat dose of 5, 20, 50, 75, 100, 200, 400, 750, or 1500 mg.

In some embodiments, at least one dose of the anti-OX40 antibody is administered at a weight-based dose of about 0.01, 0.03, 0.25, 0.1, 0.3, 1, 3, 5, 8, or 10 mg/kg body weight. In some embodiments, at least one dose of the anti-PD-1 antibody is administered at a weight-based dose of about 0.1, 0.3, 1, 3, 5, 8, or 10 mg/kg body weight. In some embodiments, at least one dose of the anti-CTLA-4 antibody is administered at a weight-based dose of about 0.1, 0.3, 1, 3, 5, 8, or 10 mg/kg body weight.

Accordingly, in some embodiments, the dose of the anti-OX40 antibody, the anti-PD-1 antibody, and/or the anti-CTLA-4 antibody is calculated per body weight, e.g., mg/kg body weight. In other embodiments, the dose of the anti-OX40 antibody, the anti-PD-1 antibody, and/or the anti-CTLA-4 antibody is a flat-fixed dose. In some embodiments, the dose of the anti-OX40 antibody, the anti-PD-1 antibody, and/or the anti-CTLA-4 antibody is varied over time. For example, in certain embodiments, the anti-OX40 antibody, the anti-PD-1 antibody, and/or the anti-CTLA-4 antibody can be initially administered at a high dose and then lowered over time. In other embodiments, the anti-OX40 antibody, the anti-PD-1 antibody, and/or the anti-CTLA-4 antibody can be initially administered at a low dose and increased over time.

In some embodiments, the amount of the anti-OX40 antibody, the anti-PD-1 antibody, and/or the anti-CTLA-4 antibody administered to a subject is constant for each dose. In other embodiments, the amount of the anti-OX40 antibody, the anti-PD-1 antibody, and/or the anti-CTLA-4 antibody administered to a subject varies with each dose. For example, the maintenance (or follow-on) dose of the antibody can be higher or the same as the loading dose which is first administered. In other embodiments, the maintenance dose of the antibody can be lower or the same as the loading dose.

In some embodiments, the anti-OX40 antibody, the anti-PD-1 antibody, and the anti-CTLA-4 antibody are formulated for intravenous administration.

In some embodiments, the anti-OX40 antibody, the anti-PD-1 antibody, and/or the anti-CTLA-4 antibody are administered once per week, once every two weeks, once every three weeks, once every four weeks, once a month, once every three months, once every four months, or once every three to six months. In some embodiments, the anti-OX40 antibody, the anti-PD-1 antibody, and/or the anti-CTLA-4 antibody are administered as long as a clinical benefit is observed or until there is a complete response, confirmed progressive disease, or unmanageable toxicity.

In some embodiments, a cycle of administration is 1, 2, 3, 4, 8, 12, 16, 20, or 24 weeks, which can be repeated as necessary. In certain embodiments, a cycle of administration is 1 week. In other embodiments, a cycle of administration is 2 weeks. In further embodiments, a cycle of administration is 3 weeks. In some embodiments, a cycle of administration is 4 weeks. In other embodiments, a cycle of administration is 8 weeks. In certain embodiments, a cycle of administration is 12 weeks. In some embodiments, a cycle of administration is 16 weeks. In additional embodiments, a cycle of administration is 20 weeks. In certain embodiments, a cycle of administration is 24 weeks. In some embodiments, doses of the anti-OX40 antibody, the anti-PD-1 antibody, and the anti-CTLA-4 antibody are administered on the same day of each administration cycle.

In some embodiments, a treatment disclosed herein consists of up to 2, 3, 4, 5, 6, 7, 8, 9, or 10 cycles. In certain embodiments, a treatment consists of 2 cycles. In other embodiments, a treatment consists of 3 cycles. In some embodiments, a treatment consists of 4 cycles. In further embodiments, a treatment consists of 5 cycles. In some embodiments, a treatment consists of 6 cycles. In further embodiments, a treatment consists of 7 cycles. In additional embodiments, a treatment consists of 8 cycles. In some embodiments, a treatment consists of 9 cycles. In certain embodiments, a treatment consists of 10 cycles.

In some embodiments, the anti-OX40 antibody, the anti-PD-1 antibody, and the anti-CTLA-4 antibody are administered as a first line of treatment (e.g., the initial or first treatment). In other embodiments, the anti-OX40 antibody, the anti-PD-1 antibody, and the anti-CTLA-4 antibody are administered as a second line of treatment (e.g., after the initial or first treatment, including after relapse and/or where the first treatment has failed).

### VIII. Outcomes

With respect to target lesions, responses to the methods disclosed herein can include:

| | |
|---|---|
| Complete Response (CR) | Disappearance of all target lesions. Any |
| (RECIST V1.1) | pathological lymph nodes (whether target |
| | or non-target) must have reduction in short |
| | axis to < 10 mm. |
| Partial Response (PR) | At least a 30% decrease in the sum of the |
| (RECIST V1.1) | diameters of target lesions, taking as |
| | reference the baseline sum diameters. |
| Progressive Disease (PD) | At least a 20% increase in the sum of the |
| (RECIST V1.1) | diameters of target lesions, taking as |
| | reference the smallest sum on study (this |
| | includes the baseline sum if that is the |
| | smallest on study). In addition to the |
| | relative increase of 20%, the sum must also |
| | demonstrate an absolute increase of at least |
| | 5 mm. (Note: the appearance of one or |
| | more new lesions is also considered |
| | progression). |
| Stable Disease (SD) | Neither sufficient shrinkage to qualify for |
| (RECIST V1.1) | PR nor sufficient increase to qualify for |
| | PD, taking as reference the smallest sum |
| | diameters while on study. |
| Immune-related | Disappearance of all target lesions. Any |
| Complete Response (irCR) | pathological lymph nodes (whether target |
| (irRECIST) | or non-target) must have reduction in short |
| | axis to < 10 mm. |
| Immune-related | At least a 30% decrease in the sum of |
| Partial Response (irPR) | diameters of target lesions and all new |
| (irRECIST) | measurable lesions (i.e., Percentage |
| | Change in Tumor Burden), taking as |
| | reference the baseline sum diameters. Note: |
| | the appearance of new measurable lesions |
| | is factored into the overall Tumor Burden, |
| | but does not automatically qualify as |
| | progressive disease until the sum of the |
| | diameters increases by ≥ 20% when |
| | compared to nadir. |
| Immune-related | At least a 20% increase in Tumor Burden |
| Progressive Disease (irPD) | (i.e., the sum of diameters of target lesions, |
| (irRECIST) | and any new measurable lesions) taking as |
| | reference the smallest sum on study (this |
| | includes the baseline sum if that is the |
| | smallest on study). In addition to the |
| | relative increase of 20%, the sum must also |
| | demonstrate an absolute increase of at least |
| | 5 mm. Tumor assessments using immune- |
| | related criteria for progressive disease |
| | incorporates the contribution of new |
| | measurable lesions. Each net percentage |
| | change in tumor burden per assessment |
| | accounts for the size and growth kinetics of |
| | both old and new lesions as they appear. |
| Immune-related | Neither sufficient shrinkage to qualify for |
| Stable Disease (irSD) | irPR nor sufficient increase to qualify for |
| (irRECIST) | irPD, taking as reference the smallest sum |
| | diameters while on study. |

With respect to non-target lesions, responses to the combination therapy disclosed herein can include:

| | |
|---|---|
| Complete Response (CR) | Disappearance of all non-target lesions. |
| (RECIST V1.1) | All lymph nodes must be non-pathological |
| | in size (<10 mm short axis). |
| Non-CR/Non-PD | Persistence of one or more non-target |
| (RECIST V1.1) | lesion(s). |
| Progressive Disease (PD) | Unequivocal progression of existing non- |
| (RECIST V1.1) | target lesions. The appearance of one or |
| | more new lesions is also considered |
| | progression. |
| Immune-related | Disappearance of all non-target lesions. All |
| Complete Response (irCR) | lymph nodes must be non-pathological in |
| (irRECIST) | size (<10 mm short axis). |
| Immune-related | Increases in number or size of non-target |
| Progressive Disease (irPD) | lesion(s) does not constitute progressive |
| (irRECIST) | disease unless/until Tumor Burden |
| | increases by 20% (i.e., the sum of the |
| | diameters at nadir of target lesions and any |
| | new measurable lesions increases by the |
| | required amount). Non-target lesions are |
| | not considered in the definition of Stable |
| | Disease and Partial Response. |

Patients treated according to the methods disclosed herein preferably experience improvement in at least one sign of cancer. In some embodiments, improvement is measured by a reduction in the quantity and/or size of measurable tumor lesions. In other embodiments, lesions can be measured on chest x-rays or CT or MRI films. In further embodiments, cytology or histology can be used to evaluate responsiveness to a therapy.

In some embodiments, the patient treated exhibits a complete response (CR), a partial response (PR), stable disease (SD), immune-related complete disease (irCR), immune-related partial response (irPR), or immune-related stable disease (irSD). In some embodiments, the patient treated experiences tumor shrinkage and/or decrease in growth rate, i.e., suppression of tumor growth. In other embodiments, the frequency of effector T cells (e.g., tumor-specific CD4⁺ or CD8⁺ effector T cells) is increased, e.g., in the tumor of a subject. In certain embodiments, the frequency of regulatory T cells (e.g., Foxp3⁺) and/or the frequency of granulocytic myeloid-derived suppressor cells (gMDSCs) is reduced. In some embodiments, unwanted cell proliferation is reduced or inhibited. In yet other embodiments, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent. In some embodiments, the methods disclosed herein produce a comparable clinical benefit rate (CBR=CR+PR+SD≥6 months) better than that achieved by an anti-OX40 antibody, an anti-PD-1 antibody, or an anti-CTLA-4 antibody alone. In other embodiments, the improvement of clinical benefit rate is about 20% 20%, 30%, 40%, 50%, 60%, 70%, 80% or more compared to an anti-OX40 antibody, an anti-PD-1 antibody, or an anti-CTLA-4 antibody alone.

In some embodiments, the tumors of the patient treated increase proinflammatory cytokine production. Inflammation is generally characterized by the interplay between pro- and anti-inflammatory cytokines. Proinflammatory cytokines include, but are not limited to interleukin-1 (IL-1), tumor necrosis factor (TNF), gamma-interferon (IFN-gamma), IL-12, IL-18 and granulocyte-macrophage colony stimulating factor, while anti-inflammatory cytokines include, but are not limited to IL-4, IL-10, IL-13, IFN-alpha and transforming growth factor-beta.

### IX. Kits and Unit Dosage Forms

Also provided herein are kits which include a pharmaceutical composition comprising an anti-OX40 antibody, an anti-PD-1 antibody, an anti-CTLA-4 antibody, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a solid tumor). The kit also can include a syringe.

Optionally, the kits include multiple packages of the single-dose pharmaceutical compositions each containing an effective amount of the anti-OX40 antibody, the anti-PD-1 antibody, and/or the anti-CTLA-4 antibody for a single administration in accordance with the methods provided above. Instruments or devices necessary for administering the pharmaceutical composition(s) also can be included in the kits. For instance, a kit can provide one or more pre-filled syringes containing an effective amount of the anti-OX40 antibody, the anti-PD-1 antibody, and/or the anti-CTLA-4 antibody.

The following examples are merely illustrative and should not be construed as limiting the scope of this disclosure in any way as many variations and equivalents will become apparent to those skilled in the art upon reading the present disclosure.

### EXAMPLES

### Example 1 Analysis of the Pharmacodynamic Effects of ANti-OX40 Antibody Combined with Anti-PD-1 and Anti-CTLA-4 Antibodies in 4T1 Tumor Model

To evaluate the pharmacodynamics effects of the combination treatment, a 4T1 tumor model for human breast cancer was used. Briefly, female BALB/c mice (7-9 weeks old, Harlan Laboratories, Frederick, Md.) were subcutaneously implanted with 1×10⁶ 4T1 cells (American Type Culture Collection) in 0.2 mL PBS using a 1-cm syringe and a 27-gauge half-inch needle. Six days after implantation, tumors were measured with calipers two-dimensionally and tumor volume was calculated using the formula: L×(W²/2), L=length (the longer of the two measurements), W=width. Mice were then randomized into 8 groups (n=5), with each group having a mean tumor volume of approximately 100 mm³. Starting at day 6 post-implantation, each mouse was dosed twice, four days apart (Q4Dx2) (i.e., at day 6 and at day 10 post-implantation) intraperitoneally with anti-OX40 antibody (3 mg/kg), anti-PD-1 antibody (10 mg/kg), anti-CTLA-4 antibody (10 mg/kg), or isotype control antibody, alone or in combination (see Table 1). The size of the tumors was monitored at days 5, 7, and 11 post-implantation. At day 12 post-implantation, the mice were sacrificed and blood and tumors were harvested for analysis as described below.

TABLE 1
Study Design

| Group No. | Treatment | Dose (mg/kg) | Dose Schedule | Route |
|---|---|---|---|---|
| 1 | IgG1 isotype | 13 | Q4D × 2 | IP |
| | IgG2b isotype | 10 | | |
| 2 | Anti-PD-1 | 10 | Q4D × 2 | IP |
| | IgG1 isotype | 3 | | |
| | IgG2b isotype | 10 | | |
| 3 | Anti-CTLA-4 | 10 | Q4D × 2 | IP |
| | IgG1 isotype | 13 | | |
| 4 | Anti-PD-1 | 10 | Q4D × 2 | IP |
| | Anti-CTLA-4 | 10 | | |
| | IgG1 isotype | 3 | | |
| 5 | Anti-OX40 | 3 | Q4D × 2 | IP |
| | IgG1 isotype | 10 | | |
| | IgG2b isotype | 10 | | |
| 6 | Anti-OX40 | 3 | Q4D × 2 | IP |
| | Anti-PD-1 | 10 | | |
| | IgG2b isotype | 10 | | |
| 7 | Anti-OX40 | 3 | Q4D × 2 | IP |
| | Anti-CTLA-4 | 10 | | |
| | IgG1 isotype | 10 | | |
| 8 | Anti-OX40 | 3 | Q4D × 2 | IP |
| | Anti-PD-1 | 10 | | |
| | Anti-CTLA-4 | 10 | | |

### Immunomonitoring of Blood and Tumors by Flow Cytometry

Blood was obtained from the mice via cardiac puncture into syringes containing ethylenediaminetetraacetic acid (EDTA). Viable white blood cells were recovered by HISTOPAQUE®-1119 (Sigma-Aldrich, Cat. 11191) gradient separation following manufacturer's instruction. Histopaque (2 mL) was added into a 15 mL conical centrifuge tube, and anticoagulated whole blood was layered onto the top of the histopaque medium. During the centrifugation, erythrocytes were aggregated by polysucrose and rapidly sedimented. White blood cells remained at the plasma-Histopaque interface. Most extraneous platelets were removed by low speed centrifugation during the washing steps.

Tumors were removed, weighed, and processed on a GENTLEMACS OCTO DISSOCIATOR™ (Miltenyi); mouse tumor dissociation kit (Miltenyi, Catalog 130-096-730) was used for tumor processing as per manufacturer's instructions. Dead cells were removed by Histopaque gradient separation. A double gradient was formed by layering an equal volume of Histopaque®-1083 (Sigma-Aldrich, Cat. 10831) over Histopaque®-1119. Single cell suspension was carefully layered onto the upper Histopaque-1083 medium. During centrifugation, dead cells were aggregated and rapidly sedimented. Granulocytes and viable tumor cells were found at the lower Histopaque-1083/1119 interface; whereas, lymphocytes and other mononuclear cells were found at the upper medium/Histopaque-1083 interface.

Viable cells from the blood and the tumors were washed, counted, and stained for immune cell markers in three panels using the flow cytometry antibodies provided in Table 2. Foxp3 and CD206 staining was performed following permeabilization to allow for intracellular staining. Antibody fluorescence was detected by flow cytometry on the Fortessa (BD Biosciences), and the results were analyzed using FlowJo software (FlowJo, LLC).

TABLE 2
Immunomonitoring Reagents and Panel Designs

| | | | | |
|---|---|---|---|---|
| | | | | Catalog |
| Marker | Fluorophore | Clone | Vendor | No. |

Panel I—Lymphocytes (Blood and Tumor)

| | | | | |
|---|---|---|---|---|
| CD45 | PE/Cy7 | 30-F11 | eBioscience | 25-0451 |
| CD11b | BUV395 | M1/70 | BD | 563553 |
| | | | Bioscience | |
| Ly-6G | APC | 1A8 | BioLegend | 127614 |
| Ly-6C | BV605 | HK1.4 | BioLegend | 128036 |
| F4/80 | BV421 | BM8 | BioLegend | 123132 |
| MHCII | ALEXA | M5/114.15.2 | BioLegend | 107616 |
| | FLUOR ® 488 | | | |
| CD40 | PE/Cy5 | 3/23 | BioLegend | 124618 |
| NKp46 | APC/FIRE ™ 750 | 29A1.4 | BioLegend | 137632 |
| CD11c | BV785 | N418 | BioLegend | 117336 |
| PD-L1 | PE | 10F.9G2 | BioLegend | 124308 |
| CD206 | PerCP/Cy5.5 | C068C2 | BioLegend | 141716 |
| CD3 | BUV737 | 17A2 | BD | 564380 |
| | | | Bioscience | |
| CD19 | BUV737 | 1D3 | BD | 564296 |
| | | | Bioscience | |
| Rat IgG2a, κ | PE/Cy5 | RTK2758 | BioLegend | 400510 |
| Isotype Ctrl | | | | |
| Rat IgG2a, κ | PerCP/Cy5.5 | RTK2758 | BioLegend | 400532 |
| Isotype Ctrl | | | | |
| Rat IgG2b, κ | PE | RTK4530 | BioLegend | 400608 |
| Isotype Ctrl | | | | |
| Rat IgG2b, κ | ALEXA FLUO ® | RTK4530 | BioLegend | 400625 |
| Isotype Ctrl | 488 | | | |

Panel II—Myeloid Cells (Tumor)

| | | | | |
|---|---|---|---|---|
| CD45 | PerCP/Cy5.5 | 30-F11 | BioLegend | 103132 |
| CD11b | BUV395 | M1/70 | BD | 563553 |
| | | | Bioscience | |
| CD3 | BV421 | 17A2 | BioLegend | 100228 |
| CD4 | BV510 | GK1.5 | BioLegend | 100449 |
| CD8a | BV786 | 53-6.7 | BioLegend | 100750 |
| Foxp3 | PE | MF-14 | BioLegend | 126404 |
| PD1 | APC | RMP1-30 | BioLegend | 109112 |
| Ki67 | PE/Cy7 | SolA15 | eBioscience | 25-5698 |
| CD19 | BUV737 | 1D3 | BD | 564296 |
| | | | Bioscience | |
| NKp46 | APC/FIRE ™ 750 | 29A1.4 | BioLegend | 137632 |
| Granzyme B | FITC | NGZB | eBioscience | 11-8898 |

Panel III—Myeloid Cells (Blood)

| | | | | |
|---|---|---|---|---|
| CD11b | BUV395 | M1/70 | BD | 563553 |
| | | | Bioscience | |
| Ly-6G | APC | 1A8 | BioLegend | 127614 |
| Ly-6C | BV605 | HK1.4 | BioLegend | 128036 |
| CD11c | BV785 | N418 | BioLegend | 117336 |
| CD3 | PE | 17A2 | BioLegend | 100206 |
| CD45R/B220 | BUV737 | RA3-6B2 | BD | 564449 |
| | | | Bioscience | |
| CD40 | PE/Cy5 | 3/23 | BioLegend | 124618 |
| CD43 | BB515 | S7 | BD | 564646 |
| | | | Bioscience | |
| NKp46 | APC/FIRE ™ 750 | 29A1.4 | BioLegend | 137632 |
| CCR2 | BV421 | SA203G11 | BioLegend | 150605 |
| PDL1 | BV711 | 10F.9G2 | BioLegend | 124319 |
| Rat IgG2b, κ | BV421 | RTK4530 | BioLegend | 400655 |
| Isotype Ctrl | | | | |
| Rat IgG2a, κ | PE/Cy5 | RTK2758 | BioLegend | 400510 |
| Isotype Ctrl | | | | |

### Cytokine and Chemokine Analysis in Tumor Supernatants

Supernatants collected following centrifugation of the tumor single-cell suspensions were analyzed for levels of different cytokines and chemokines. Following the first centrifugation of the single-cell tumor suspensions (400×g, 10 min at 4-8° C.), supernatants were collected, and transferred on ice for immediate analysis. Luminex based methodology was used for analysis using commercial reagent kits (MCYTOMAG-70K and MCYTOMAG-73K) as per the manufacturer's standard protocol. Briefly, 25 μL of each sample was aliquoted into 96-well plates along with reference standards. The samples were then diluted with assay reagent matrix buffer and incubated overnight at 4° C. with capture antibody cocktails. Afterwards, samples were washed using a hand held 96-well plate magnet, and incubated with detection and PE secondary reagents for two hours at room temperature on an orbital shaker. Samples were read using a Luminex brand LX-200 instrument and cytokines/chemokines were quantified using 5-parameter log fit algorithms for each of the standard curves. For tumor supernatants, concentrations of each analyte were normalized to the weight of the tumor tissue to provide values as pg/mL/g of tumor.

### Results

### Antitumor Activity

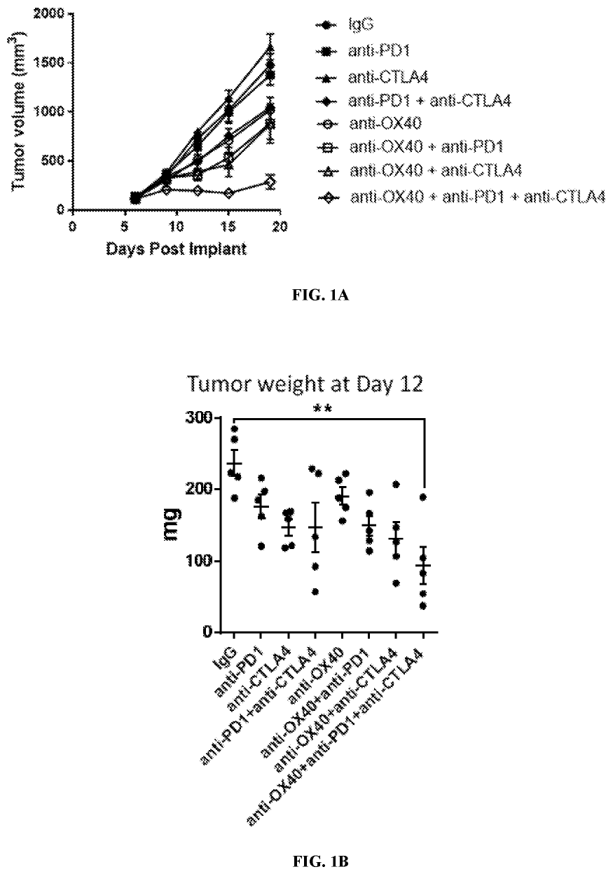

As shown in FIG. 1A, mice that received a combination of all three antibodies (anti-OX40, anti-PD-1, and anti-CTLA-4, “triple combination”) had reduced tumor volume compared to mice that received the other treatment regimens, particularly the isotype control antibody. This difference was first apparent at around day 7 post implantation and was much more pronounced at day 11. Similarly, when the mice were sacrificed at day 12 post implantation, tumor weight from mice that received the triple combination was significantly less compared to the other treatment groups. FIG. 1B.

### Immunophenotyping Analysis

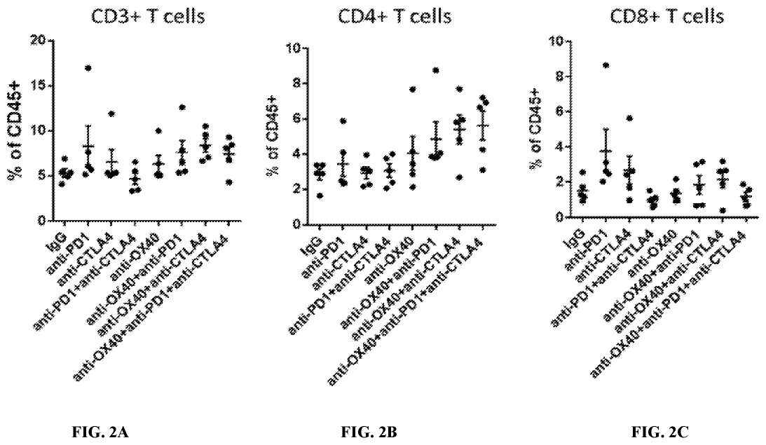

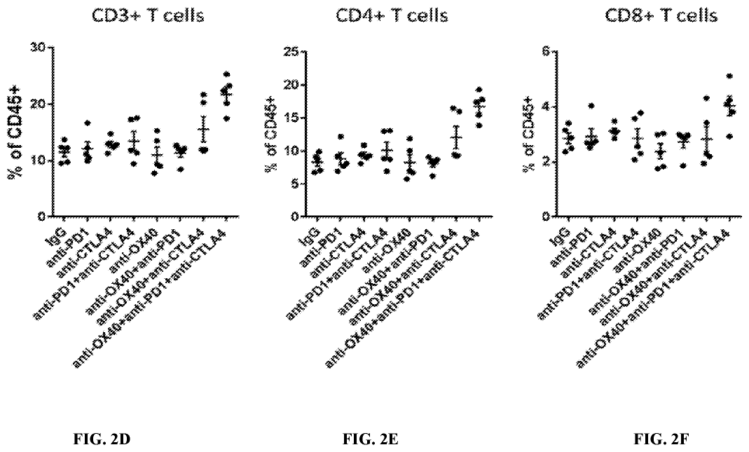

The lymphocyte populations from both blood and tumors were assessed using the phenotypic markers provided in Panel 1 of Table 2. As shown in FIGS. 2A-2C, the percentages of major T cell populations (i.e., CD3⁺ cells, CD4⁺ cells, and CD8⁺ cells) within the CD45⁺ infiltrating leukocytes were not significantly altered in the tumors from the different treatment groups. However, in the blood, mice that received the triple combination had significantly higher percentages of T cells (CD3⁺ cells, CD4⁺ cells, and CD8⁺ cells) within the CD45⁺ cell population, compared to any of the other treatment groups. FIGS. 2D-2F.

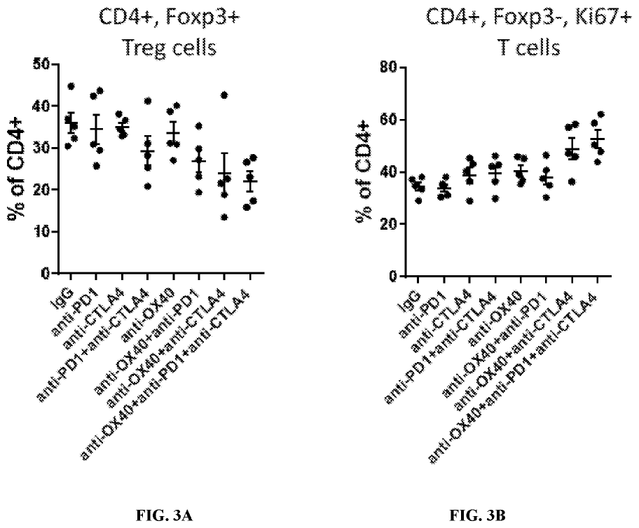

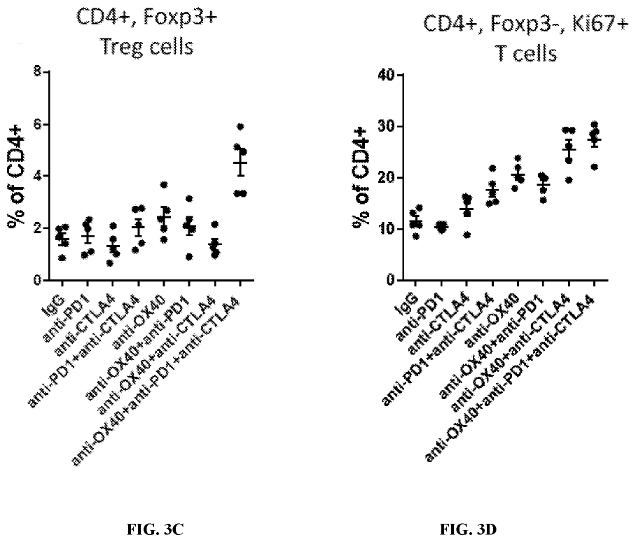

The observed increase of the CD4⁺ T cell population in peripheral blood of was associated with increases in both the proliferation of effector CD4+ T cells (Foxp3⁻ Ki67⁺) and the frequency of Foxp3⁺ regulatory T cells. FIGS. 3C and 3D. In the tumors, while there was also an increase in the frequency of proliferating effector CD4⁺ T cells (FIG. 3B), the frequency of Foxp3⁺ regulatory T cells (Tregs) decreased by more than 30% in the triple combination group, compared to the control and monotherapy treated groups (FIG. 3A). This decrease in the Treg population can possibly explain the lack of increase in the tumors when total CD4⁺ T cells were analyzed.

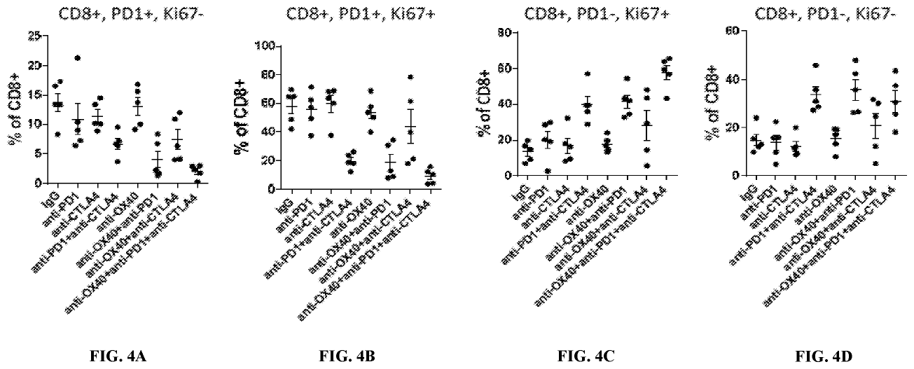

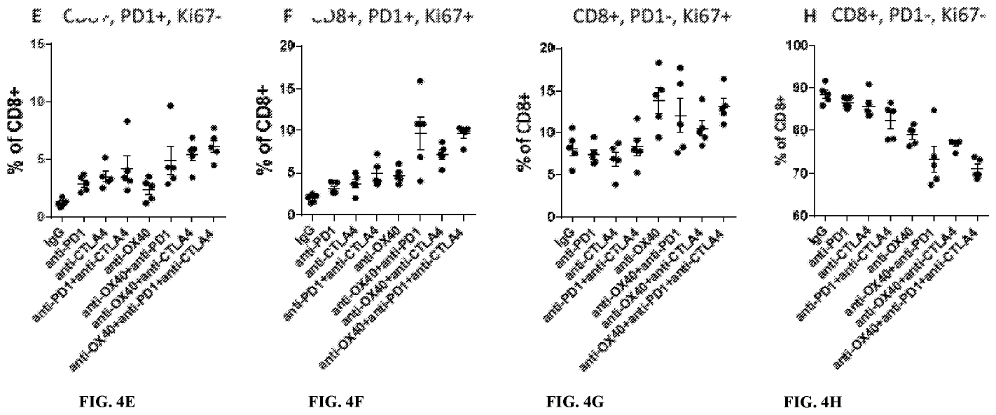

Within the CD8⁺ T cell population, the tumors from mice that received the triple combination had significantly reduced frequency of exhausted (PD-1⁺) CD8⁺ T cells (FIGS. 4A and 4B) with an increase in the frequency of proliferating effector CD8⁺ T cell population (PDF Ki67⁺) (FIG. 4C) compared to the other treatment groups. There was no observed difference within the PD1⁻Ki67⁻ CD8⁺ T cell population in the tumors, at least between the triple combination group and animals that received the dual combination of anti-PD-1 and anti-CTLA-4 antibodies. FIG. 4D. In the peripheral blood, mice that received the triple combination had increased frequency of both exhausted (PD-1⁺) CD8⁺ T cells (FIGS. 4E and 4F) and proliferating effector CD8⁺ T cells (PD-1⁻ Ki67⁺, FIG. 4G), compared to the other treatment groups. In contrast, the population of non-proliferating effector CD8 T cells (PD-1⁻ Ki67⁻) was reduced in the triple combination group compared to the other animals. FIG. 4H.

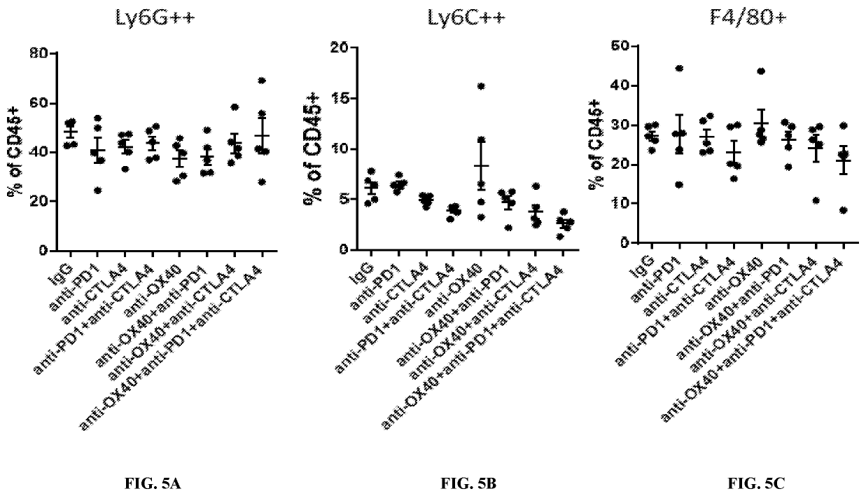

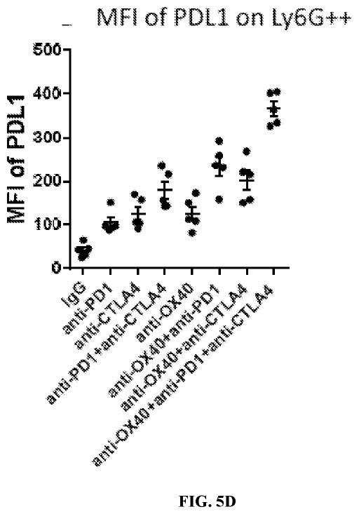

To assess the myeloid cell populations in the tumor and blood of the animals, Panels 2 and 3 of Table 2 were used, respectively. As shown in FIGS. 5A-5C, the percentages of the various myeloid cell populations in the tumor were similar amongst the treatment groups, although there appeared to be a non-significant trend for lower percentage of Ly6C⁺⁺ monocytes in the triple combination group. Moreover, there was a significant increase in PD-L1 expression on the tumor infiltrating Ly6G⁺⁺ gMDSCs in the triple combination group compared to the other animals. FIG. 5D. The induction of PD-L1 on tumor cells and myeloid cells has been reported to be dependent (or partially dependent) on IFN-γ. Noguchi, T., et al., Cancer Immunol Res 5(2): 106-117 (2017). Accordingly, this increase in PD-L1 expression suggested that there was an increase in IFN-γ in the tumor microenvironment of mice that received the triple combination, compared to the other groups.

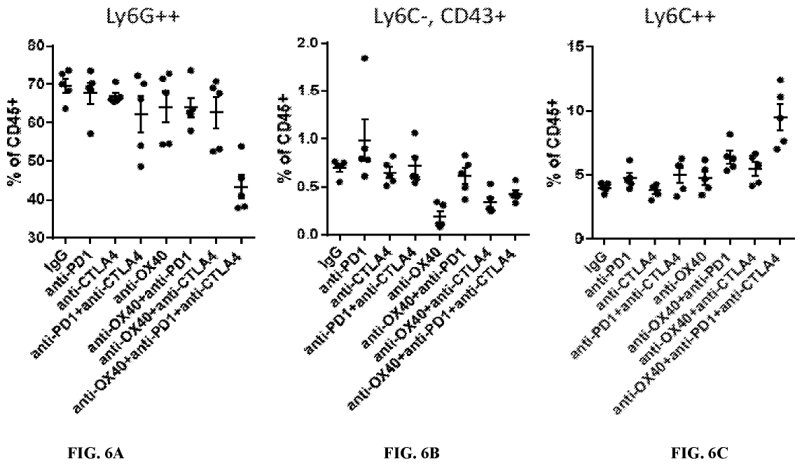

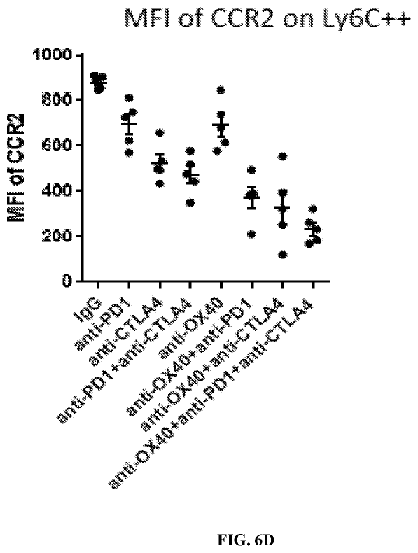

As observed with the lymphocyte populations, differences in the myeloid cell populations were more noticeable in the blood. As shown in FIGS. 6A and 6B, mice that received the triple combination had reduced frequency of Ly6G⁺⁺ gMDSCs and non-classical Ly6C⁻ CD43⁺ monocytes in the blood, respectively. While the blood from the triple combination group had increased frequency of classical Ly6C⁺⁺ monocytes (FIG. 6C), these monocytes had significantly reduced CCR2 expression, compared to the other groups (FIG. 6D). CCR2 expression has been previously shown to be important for monocyte trafficking to the tumor site. Sawanobori, Y., et al., Blood 111(12):5477-66 (2008). Accordingly, the reduced CCR2 expression can offer one possible explanation as to why there was no increase in the myeloid cell populations in the tumor, despite an increase in the frequency of classical Ly6C⁺⁺ monocytes in the blood.

### Chemokine and Cytokine Analysis

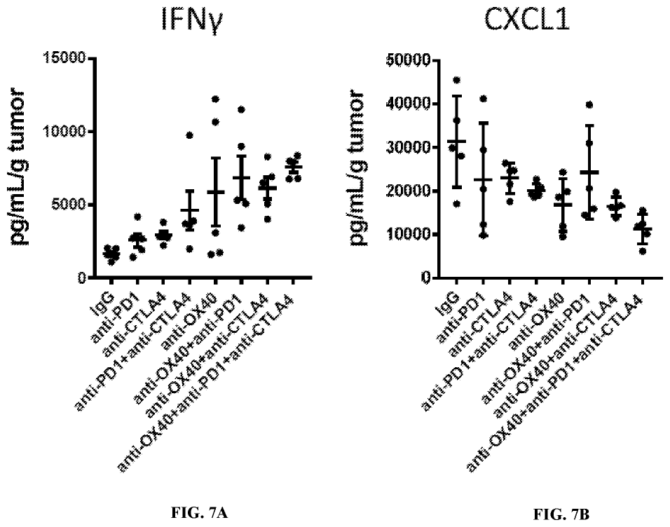

As shown in FIG. 7A, mice that received the triple combination had increased IFN-γ levels in the tumors, compared to the other treatment groups, including animals that received the dual combination of anti-PD-1 and anti-CTLA-4 antibodies. This result was consistent with the increase in PD-L1 expression observed on the Ly6G⁺⁺ gMDSCs in the tumors of the triple combination group (see FIG. 5D). Compared to the other treatment groups, mice that received the triple combination also had reduced intratumoral CXCL1 levels. CXCL1 is a chemokine which signals through CXCR2 and is important in the recruitment of immunosuppressive neutrophils and gMDSCs. This result appeared to be consistent with the observed decrease in the level of circulating gMDSCs in the triple combination-treated mice (see FIG. 6A).

Data for additional cytokines and chemokines measured in the tumor supernatants are provided in Tables 3-6.

TABLE 3
Tumor Cytokines and Chemokines (pg/mL/g tumor, n = 5)

| No. | Treatment | Mean ± SEM | IFN-γ | IL-1β | IL-2 | IL-4 | IL-5 |
|---|---|---|---|---|---|---|---|
| 1 | IgG | Mean ± SEM | 1639 ± 178 | 30023 ± 1431 | 284 ± 58 | 141 ± 22 | 304 ± 57 |
| 2 | Anti-PD-1 | Mean ± SEM | 2588 ± 467 | 20259 ± 2813 | 440 ± 96 | 149 ± 27 | 369 ± 55 |
| 3 | Anti-CTLA-4 | Mean ± SEM | 2924 ± 253 | 22455 ± 1742 | 626 ± 69 | 203 ± 23 | 1570 ± 949 |
| 4 | Anti-PD-1 + | Mean ± SEM | 4601 ± 1333 | 31556 ± 4914 | 1057 ± 325 | 359 ± 106 | 1618 ± 530 |
| | Anti-CTLA-4 | | | | | | |
| 5 | Anti-OX40 | Mean ± SEM | 5868 ± 2304 | 20173 ± 2049 | 423 ± 80 | 156 ± 18 | 1087 ± 187 |
| 6 | Anti-OX40 + | Mean ± SEM | 6863 ± 1472 | 32710 ± 1450 | 1031 ± 63 | 351 ± 65 | 952 ± 218 |
| | Anti-PD1 | | | | | | |
| 7 | Anti-OX40 + | Mean ± SEM | 6157 ± 737 | 33394 ± 2919 | 904 ± 115 | 319 ± 26 | 1761 ± 653 |
| | Anti-CTLA-4 | | | | | | |
| 8 | Anti-OX40 + | Mean ± SEM | 7567 ± 329 | 32153 ± 2175 | 1207 ± 131 | 381 ± 44 | 1452 ± 238 |
| | Anti-PD-1 + | | | | | | |
| | Anti-CTLA-4 | | | | | | |

TABLE 4
Tumor Cytokines and Chemokines (pg/mL/g tumor, n = 5)

| No. | Treatment | Mean ± SEM | IL-6 | IL-9 | IL-10 | IL-12p70 | IL-17 |
|---|---|---|---|---|---|---|---|
| 1 | IgG | Mean ± SEM | 2625 ± 247 | 2660 ± 507 | 3645 ± 1100 | 2348 ± 211 | 758 ± 90 |
| 2 | Anti-PD-1 | Mean ± SEM | 2300 ± 449 | 3778 ± 766 | 2986 ± 781 | 2308 ± 493 | 703 ± 115 |
| 3 | Anti-CTLA-4 | Mean ± SEM | 3171 ± 391 | 4527 ± 514 | 4093 ± 1113 | 3392 ± 559 | 949 ± 192 |
| 4 | Anti-PD-1 + | Mean ± SEM | 4900 ± 1191 | 6288 ± 1626 | 4780 ± 1118 | 6097 ± 1827 | 1570 ± 417 |
| | Anti-CTLA-4 | | | | | | |
| 5 | Anti-OX40 | Mean ± SEM | 2546 ± 262 | 3658 ± 554 | 2572 ± 289 | 2408 ± 353 | 724 ± 67 |
| 6 | Anti-OX40 + | Mean ± SEM | 5363 ± 513 | 5693 ± 597 | 5440 ± 631 | 6035 ± 683 | 1599 ± 244 |
| | Anti-PD1 | | | | | | |
| 7 | Anti-OX40 + | Mean ± SEM | 4862 ± 476 | 5638 ± 797 | 4793 ± 477 | 5718 ± 824 | 1530 ± 225 |
| | Anti-CTLA-4 | | | | | | |
| 8 | Anti-OX40 + | Mean ± SEM | 5484 ± 439 | 9042 ± 1380 | 5018 ± 554 | 5908 ± 847 | 1526 ± 163 |
| | Anti-PD-1 + | | | | | | |
| | Anti-CTLA-4 | | | | | | |

TABLE 5
Tumor Cytokines and Chemokines (pg/mL/g tumor, n = 5)

| No. | Treatment | Mean ± SEM | IP-10 | CXCL1 | MCP-1 | MIG | VEGF |
|---|---|---|---|---|---|---|---|
| 1 | IgG | Mean ± SEM | 878 ± 174 | 31378 ± 4687 | 26605 ± 2167 | 20759 ± 1821 | 873 ± 117 |
| 2 | Anti-PD-1 | Mean ± SEM | 866 ± 47 | 22684 ± 5785 | 27843 ± 5275 | 24634 ± 3144 | 2646 ± 746 |
| 3 | Anti-CTLA-4 | Mean ± SEM | 800 ± 107 | 22993 ± 1563 | 27308 ± 2607 | 25641 ± 3481 | 5378 ± 607 |
| 4 | Anti-PD-1 + | Mean ± SEM | 1053 ± 370 | 20137 ± 727 | 19819 ± 1546 | 24205 ± 5421 | 5651 ± 1255 |
| | Anti-CTLA-4 | | | | | | |
| 5 | Anti-OX40 | Mean ± SEM | 1200 ± 246 | 16862 ± 2714 | 21027 ± 1684 | 26895 ± 1746 | 2241 ± 897 |
| 6 | Anti-OX40 + | Mean ± SEM | 940 ± 131 | 24390 ± 4827 | 25924 ± 3807 | 30119 ± 3485 | 4354 ± 522 |
| | Anti-PD1 | | | | | | |
| 7 | Anti-OX40 + | Mean ± SEM | 1000 ± 230 | 16557 ± 953 | 26192 ± 3339 | 24741 ± 3759 | 4449 ± 871 |
| | Anti-CTLA-4 | | | | | | |
| 8 | Anti-OX40 + | Mean ± SEM | 943 ± 135 | 11265 ± 1536 | 19800 ± 2886 | 24920 ± 1673 | 4690 ± 795 |
| | Anti-PD-1 + | | | | | | |
| | Anti-CTLA-4 | | | | | | |

TABLE 6
Tumor Cytokines and Chemokines (pg/mL/g tumor, n = 5)

| No. | Treatment | Mean ± SEM | TNF-α | MCP5 | TARC | IL-21 |
|---|---|---|---|---|---|---|
| 1 | IgG | Mean ± SEM | 1813 ± 243 | 14311 ± 6572 | 1026 ± 177 | 11979 ± 1416 |
| 2 | Anti-PD-1 | Mean ± SEM | 1268 ± 192 | 96031 ± 25645 | 674 ± 63 | 14290 ± 3502 |
| 3 | Anti-CTLA-4 | Mean ± SEM | 2023 ± 353 | 47031 ± 7215 | 456 ± 201 | 9375 ± 2130 |
| 4 | Anti-PD-1 + | Mean ± SEM | 2629 ± 672 | 30746 ± 6310 | 844 ± 371 | 29077 ± 12430 |
| | Anti-CTLA-4 | | | | | |
| 5 | Anti-OX40 | Mean ± SEM | 1706 ± 240 | 118792 ± 32410 | 1771 ± 514 | 16692 ± 2926 |
| 6 | Anti-OX40 + | Mean ± SEM | 2871 ± 309 | 39122 ± 6899 | 2273 ± 269 | 44574 ± 6748 |
| | Anti-PD1 | | | | | |
| 7 | Anti-OX40 + | Mean ± SEM | 2793 ± 241 | 74024 ± 36311 | 2059 ± 619 | 23711 ± 3726 |
| | Anti-CTLA-4 | | | | | |
| 8 | Anti-OX40 + | Mean ± SEM | 3288 ± 299 | 35379 ± 4049 | 1919 ± 217 | 51138 ± 9065 |
| | Anti-PD-1 + | | | | | |
| | Anti-CTLA-4 | | | | | |

Collectively, the above data demonstrated that the combination of anti-OX40, anti-PD-1, and anti-CTLA-4 antibodies leads to a significant enhancement in therapeutic efficacy over other treatment options tested herein (e.g., a dual therapy of anti-PD-1 and anti-CTLA-4 antibodies) in a mouse 4T1 breast tumor model. The increased anti-tumor efficacy was accompanied by changes in both the peripheral and intratumoral leukocyte populations, as well as cytokine milieu, that would favor a more productive anti-tumor immune response, relief of immunosuppression by gMDSCs and Tregs, increased CD4⁺ and CD8⁺ effector T cell populations, and/or decreased exhausted T cells.

### Example 2 Analysis of the Antitumor Efficacy of Anti-OX40 Antibody Combined with Anti-PD-1 and Anti-CTLA-4 Antibodies in 4T1 Tumor Model

To study the effect of the triple combination therapy on tumor growth, a 4T1 tumor model was again used as described in Example 1. After tumor implantation, mice were randomized into 16 groups (n=10), with each group having a mean tumor volume of approximately 100 mm³. Starting at day 6 post-implantation, mice were then dosed four times, four days apart between each dose, (Q4D ×4) (i.e., days 6, 10, 14, and 18 post-implantation) with anti-OX40 antibody, anti-PD-1 antibody, anti-CTLA-4 antibody, or isotype control antibody, alone or in combination, as shown in Table 7.

TABLE 7
Study Design

| Group | | Dose | Dose | |
|---|---|---|---|---|
| No. | Treatment | (mg/kg) | Schedule | Route |
| 1 | IgG1 isotype | 20 | Q4D × 4 | IP |
| | IgG2b isotype | 10 | | |
| 2 | Anti-PD-1 | 10 | Q4D × 4 | IP |
| | IgG1 isotype | 10 | | |
| | IgG2b isotype | 10 | | |
| 3 | Anti-CTLA-4 | 10 | Q4D × 4 | IP |
| | IgG1 isotype | 20 | | |
| 4 | Anti-PD-1 | 10 | Q4D × 4 | IP |
| | Anti-CTLA-4 | 10 | | |
| | IgG1 isotype | 10 | | |
| 5 | Anti-OX40 | 10 | Q4D × 4 | IP |
| | IgG1 isotype | 10 | | |
| | IgG2b isotype | 10 | | |
| 6 | Anti-OX40 | 1 | Q4D × 4 | IP |
| | IgG1 isotype | 19 | | |
| | IgG2b isotype | 10 | | |
| 7 | Anti-OX40 | 0.1 | Q4D × 4 | IP |
| | IgG1 isotype | 19.9 | | |
| | IgG1 isotype | 10 | | |
| 8 | Anti-OX40 | 10 | Q4D × 4 | IP |
| | Anti-PD-1 | 10 | | |
| | IgG2b isotype | 10 | | |
| 9 | Anti-OX40 | 10 | Q4D × 4 | IP |
| | Anti-CTLA-4 | 10 | | |
| | IgG1 isotype | 10 | | |
| 10 | Anti-OX40 | 10 | Q4D × 4 | IP |
| | Anti-PD-1 | 10 | | |
| | Anti-CTLA-4 | 10 | | |
| 11 | Anti-OX40 | 1 | Q4D × 4 | IP |
| | Anti-PD-1 | 10 | | |
| | IgG1 isotype | 9 | | |
| | IgG2b isotype | 10 | | |
| 12 | Anti-OX40 | 1 | Q4D × 4 | IP |
| | Anti-CTLA-4 | 10 | | |
| | IgG1 isotype | 19 | | |
| 13 | Anti-OX40 | 1 | Q4D × 4 | IP |
| | Anti-PD-1 | 10 | | |
| | Anti-CTLA-4 | 10 | | |
| | IgG1 isotype | 9 | | |
| 14 | Anti-OX40 | 0.1 | Q4D × 4 | IP |
| | Anti-PD-1 | 10 | | |
| | IgG1 isotype | 9.9 | | |
| | IgG2b isotype | 10 | | |
| 15 | Anti-OX40 | 0.1 | Q4D × 4 | IP |
| | Anti-CTLA-4 | 10 | | |
| | IgG1 isotype | 19.9 | | |
| 16 | Anti-OX40 | 0.1 | Q4D × 4 | IP |
| | Anti-PD-1 | 10 | | |
| | Anti-CTLA-4 | 10 | | |
| | IgG1 isotype | 9.9 | | |

Animals were checked daily for postural, grooming, and respiratory changes, as well as lethargy and tumor ulceration. Animals were weighed at least twice weekly and euthanized if weight loss was ≥20%. The flanks of each animal were also checked for the presence and size of tumors at least twice weekly until death, euthanasia, or end of the study period. Tumor volume was calculated using the formula: L×(W²/2), L=length (the longer of the two measurements), W=width. Anti-tumor efficacy was expressed as percentage tumor growth inhibition (% TGI), calculated as follows: % TGI={1−[(Tt−To)/(Ct−Co)]}×100, where Ct=the median tumor volume (mm³) of vehicle control (C) treated mice at time t; Tt=median tumor volume (mm³) of treated mice at time t; To=median tumor size of treated group at treatment initiation; Co=median tumor size at treatment initiation. When the tumor reached approximately 1500 mm³ or appeared ulcerated, animals were euthanized. Furthermore, if the tumor was expected to reach the maximum allowed volume (1500 mm³) the same day, the mouse was euthanized.

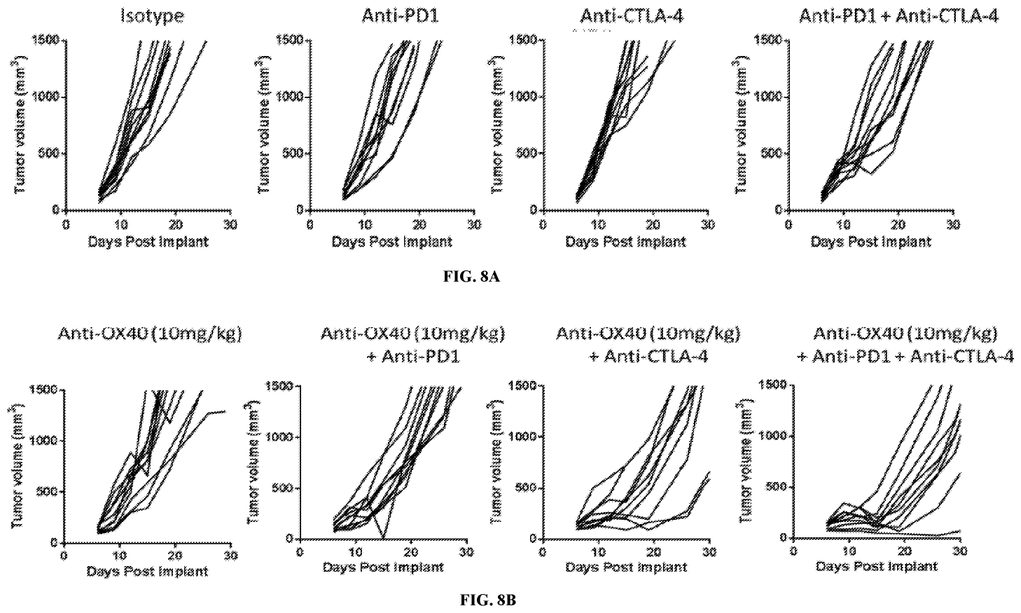

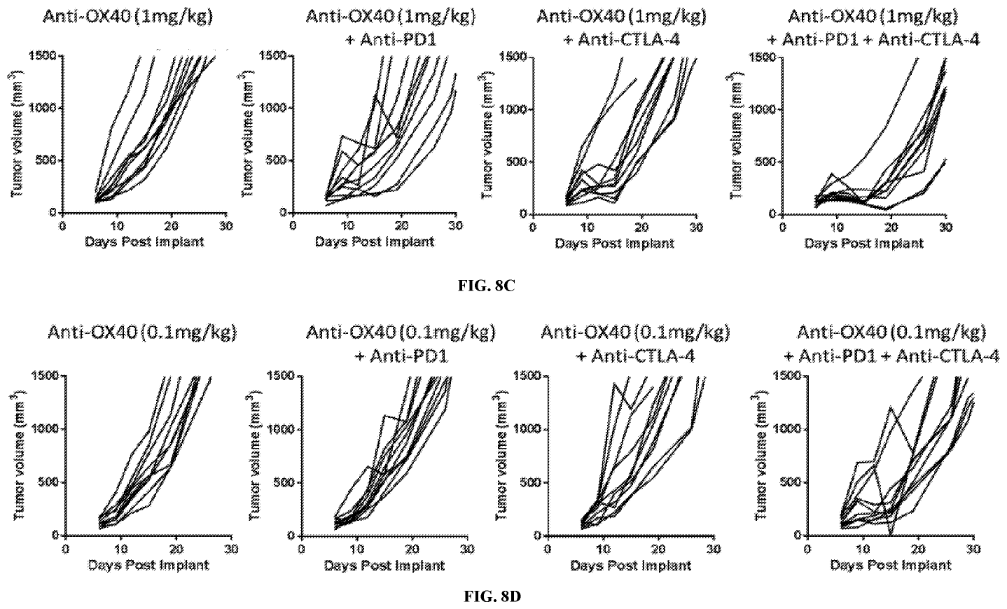

As shown in FIG. 8A, monotherapy treatment with either anti-PD1 antibody or anti-CTLA-4 antibody failed to control tumor growth, resulting in a median survival of 19 days (Table 8). Animals that received anti-OX40 antibody alone, at either 0.1 mg/kg or 1 mg/kg, had a mean survival of 26 days, suggesting that as a monotherapy (Table 8), the anti-OX40 antibody can be more efficacious.

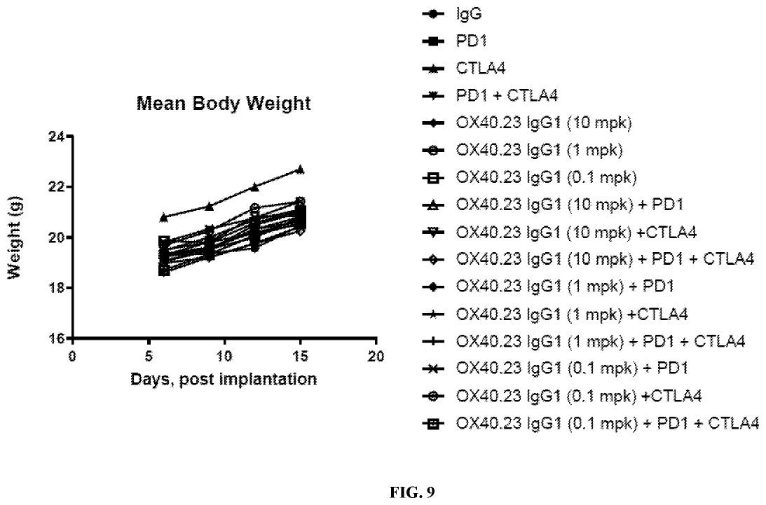

As a combination therapy, the administration of all three antibodies (i.e., anti-OX40, anti-PD-1, and anti-CTLA-4) had the greatest antitumor effect on the animals. Table 8 and FIGS. 8B-8D. For instance, the dual combination of anti-PD-1 and anti-CTLA-4 antibodies resulted in a median tumor growth inhibition of 9.8% with a median survival of 26 days. Table 8. When these antibodies were administered to the mice with the anti-OX40 antibody (e.g., 1 mg/kg), the median tumor growth inhibition increased to 84.4% and the median survival increased to 33 days. Slightly reduced antitumor efficacy was observed when the anti-OX40 antibody was administered at 10 mg/kg or 0.1 mg/kg in combination with the anti-PD-1 and anti-CTLA-4 antibodies. No body weight losses were seen as a result of any of the treatments. FIG. 9.

TABLE 8
Tumor Growth Inhibition (TGI) and Median Survival by
Treatment Group

| | | Median | | | |
|---|---|---|---|---|---|
| | | Tumor | Median | Median | |
| Group | | Volume | TGI | Survival | |
| No. | Treatment | (mm³) | (%) | (Days) | P value |
| 1 | Isotype | 1447.5 | N/A | 19 | N/A |
| 2 | Anti-PD-1 | 1458.3 | 12.5 | 19 | 0.9164 |
| 3 | Anti-CTLA-4 | 1820 | −49.0 | 19 | 0.8163 |
| 4 | Anti-CTLA-4 + | 954.6 | 9.8 | 26 | 0.0003 |
| | Anti-PD-1 | | | | |
| 5 | Anti-OX40 | 1461.1 | 2.06 | 22.5 | 0.995 |
| | (10 mg/kg) | | | | |
| 6 | Anti-OX40 | 916.9 | 24.3 | 26 | 0.0011 |
| | (1 mg/kg) | | | | |
| 7 | Anti-OX40 | 867.2 | 30.8 | 26 | 0.0001 |
| | (0.1 mg/kg) | | | | |
| 8 | Anti-OX40 | 759.8 | 44.2 | 26 | 0.0001 |
| | (10 mg/kg) + | | | | |
| | Anti-PD-1 | | | | |
| 9 | Anti-OX40 | 537.9 | 61.3 | 29 | 0.0001 |
| | (10 mg/kg) + | | | | |
| | Anti-CTLA-4 | | | | |
| 10 | Anti-OX40 | 302.3 | 83.6 | 33 | 0.0001 |
| | (10 mg/kg) + | | | | |
| | Anti-CTLA-4 + | | | | |
| | Anti-PD-1 | | | | |
| 11 | Anti-OX40 | 696.5 | 60.7 | 26 | 0.0001 |
| | (1 mg/kg) + | | | | |
| | Anti-PD-1 | | | | |
| 12 | Anti-OX40 | 756.2 | 39.9 | 26 | 0.0001 |
| | (1 mg/kg) + | | | | |
| | Anti-CTLA-4 | | | | |
| 13 | Anti-OX40 | 260.5 | 84.4 | 33 | 0.0001 |
| | (1 mg/kg) + | | | | |
| | Anti-CTLA-4 + | | | | |
| | Anti-PD-1 | | | | |
| 14 | Anti-OX40 | 919.4 | 42.5 | 26 | 0.0001 |
| | (0.1 mg/kg) + | | | | |
| | Anti-PD-1 | | | | |
| 15 | Anti-OX40 | 1076.6 | 14.6 | 26 | 0.0061 |
| | (0.1 mg/kg) + | | | | |
| | Anti-CTLA-4 | | | | |
| 16 | Anti-OX40 | 672.6 | 56.3 | 29 | 0.0001 |
| | (0.1 mg/kg) + | | | | |
| | Anti-CTLA-4 + | | | | |
| | Anti-PD-1 | | | | |

Collectively, the above data suggest that the triple combination therapy described herein can be a viable treatment option for certain cancers, including those where dual combination therapy approaches (e.g., anti-PD-1 and anti-CTLA-4 antibodies) have had limited efficacy.

TABLE 9
Listing of Sequences

| SEQ ID | Description | Sequences |
|---|---|---|
| 1 | 3F4 VH CDR1 | SYDVN |
| 2 | 3F4 VH CDR2 | WMNPNSGNTGYAPKFQG |
| 3 | 3F4 VH CDR3 | IYSSSYNWFDP |
| 4 | 3F4 VL CDR1 | RASQSVSSYLA |
| 5 | 3F4 VL CDR2 | DASNRAT |
| 6 | 3F4 VL CDR3 | QQRSNWPLT |
| 7 | 3F4 VH | QVQLVQSGAEVKKPGASVKVSCKASGNTFTSYDVNWVRQATGQ |
| | | GLEWMGWMNPNSGNTGYAPKFQGRVTMTRNTSISTAYMELSSL |
| | | RSEDTAVYYCARIYSSSYNWFDPWGQGTLVTVSS |
| 8 | 3F4 VL | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPLTFGGGTKVEIK |
| 9 | 14B6 VH CDR1 | SNWIG |
| 10 | 14B6 VH CDR2 | FIYPGDSDTRYSPSFQG |
| 11 | 14B6 VH CDR3 | YGDDWYFDL |
| 12 | 14B6 VL1 CDR1 | RASQSVSSYLA |
| 13 | 14B6 VL1 CDR2 | DASNRAT |
| 14 | 14B6 VL1 CDR3 | QQRGDWPIT |
| 15 | 14B6 VL2 CDR1 | RASQGISSWLA |
| 16 | 14B6 VL2 CDR2 | AASSLQS |
| 17 | 14B6 VL2 CDR3 | QQYNSYPRIT |
| 18 | 14B6 VH | EVQLEQSGAEVKKPGESLKISCKGSGYSFTSNWIGWVRQMPGK |
| | | GLEWMGFIYPGDSDTRYSPSFQGQVTISADKSISTAYLQWSSL |
| | | KASDIAMYYCARYGDDWYFDLWGRGTLVTVSS |
| 19 | 14B6 VL1 | EIVLTQSPATLSLSPGERATLSCRASQSVSSYLAWFQQRPGQA |
| | | PRLLIYDASNRATGIPARFSGSGSGTDFSLTISSLEPEDFAVY |
| | | YCQQRGDWPITFGQGTRLEIK |
| 20 | 14B6 VL2 | DIQMTQSPSSLSASVGDRVTITCRASQGISSWLAWYQQKPEKA |
| | | PKSLIYAASSLQSGVPSRFSGSGSGTDFTLTISSLQPEDFATY |
| | | YCQQYNSYPRITFGQGTRLEIK |
| 21 | 23H3 VH CDR1 | NYAMY |
| 22 | 23H3 VH CDR2 | AIGIGGDTFYTDSVKG |
| 23 | 23H3 VH CDR3 | MGTGYFFDY |
| 24 | 23H3 VL CDR1 | RASQSVSSYLA |
| 25 | 23H3 VL CDR2 | DASNRAT |
| 26 | 23H3 VL CDR3 | QQRSNWPLT |
| 27 | 23H3 VH | EVQLVQSGGGLVHPGGSLRLSCAGSGFTFSNYAMYWVRQAPGK |
| | | GLEWVSAIGIGGDTFYTDSVKGRFTISRDNAKNSLSLQMNSLR |
| | | AEDMAVYYCARMGTGYFFDYWGQGTLVTVSS |
| 28 | 23H3 VL | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPLTFGPGTKVDIK |
| 29 | 6E1 VH CDR1 | SFAMH |
| 30 | 6E1 VH CDR2 | VISYDGSIKYYTDSVKG |
| 31 | 6E1 VH CDR3 | DGNYGSARYFQH |
| 32 | 6E1 VL1 CDR1 | RASQGISSWLA |
| 33 | 6E1 VL1 CDR2 | AASSLQS |
| 34 | 6E1 VL1 CDR3 | QQYNSYPRT |
| 35 | 6E1 VL2 CDR1 | RASQSVSSYLA |
| 36 | 6E1 VL2 CDR2 | DASNRAT |
| 37 | 6E1 VL2 CDR3 | QQRSNWPYT |
| 38 | 6E1 VH | QVQLVESGGGVVQPGRSLRLSCAASGFTFSSFAMHWVRQAPGK |
| | | GLEWVTVISYDGSIKYYTDSVKGRFTFSRDNSKNTLYLQMNSL |
| | | RAEDTAVYYCTRDGNYGSARYFQHWGQGTLVTVSS |
| 39 | 6E1 VL1 | DIQMTQSPSSLSASVGDRVTITCRASQGISSWLAWYQQKPEKA |
| | | PKSLIYAASSLQSGVPSRFSGSGSGTDFTLTISSLQPEDFATY |
| | | YCQQYNSYPRTFGQGTKVEIK |
| 40 | 6E1 VL2 | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPYTFGQGTKLEIK |
| 41 | 18E9 VH CDR1 | SSAMH |
| 42 | 18E9 VH CDR2 | AIGTGGDTYYADSVKG |
| 43 | 18E9 VH CDR3 | DFYDILTGIFDY |
| 44 | 18E9 VL CDR1 | RASQGISSWLA |
| 45 | 18E9 VL CDR2 | AASSLQS |
| 46 | 18E9 VL CDR3 | QQANSFPST |
| 47 | 18E9 VH | EVQLVQSGGGLVHPGGSLRLSCAHSGFTFTSSAMHWVRQAPGK |
| | | GLEWISAIGTGGDTYYADSVKGRFTISRDNAKNSLYLQINSLR |
| | | AEDMAVYYCARDFYDILTGIFDYWGQGTLVTVSS |
| 48 | 18E9 VL | DIQMTQSPSSVSASVGDRVTITCRASQGISSWLAWYQHKPGKA |
| | | PKLLIYAASSLQSGVPSRFSGSGSGTDFTLTISSLQPEDFATY |
| | | YCQQANSFPSTFGQGTKVEIK |
| 49 | 8B11 VH CDR1 | SDAMY |
| 50 | 8B11 VH CDR2 | AIGIGGDTYYTDSVMG |
| 51 | 8B11 VH CDR3 | LGMGYYFDY |
| 52 | 8B11 VL CDR1 | RASQSVSSYLA |
| 53 | 8B11 VL CDR2 | DASNRAT |
| 54 | 8B11 VL CDR3 | QQRSNWPPT |
| 55 | 8B11 VH | MEFVLSWVFLVAILKGVQCEIQLVQSGGGLVHPGGSLRLSCAG |
| | | SGFTFSSDAMYWVRQAPGKGLEWVSAIGIGGDTYYTDSVMGRF |
| | | TISRDNAKNSLYLQMNSLRAEDMAVYYCARLGMGYYFDYWGQG |
| | | TLVTVSS |
| 56 | 8B11 VL | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPPTFGQGTKVEIK |
| 57 | 20B3 VH CDR1 | SYDMH |
| 58 | 20B3 VH CDR2 | VIGTAGDTYYPGSVKG |
| 59 | 20B3 VH CDR3 | GGMGNYFDY |
| 60 | 20B3 VL CDR1 | RASQSVSSYLA |
| 61 | 20B3 VL CDR2 | DASNRAT |
| 62 | 20B3 VL CDR3 | QQRSNWPLT |
| 63 | 20B3 VH | EVQLVESGGGLVQPGGSLRLSCAASGFTFSSYDMHWVRQTTGK |
| | | GLEWVSVIGTAGDTYYPGSVKGRFTISRENAKNSLYLQMNSLR |
| | | AGDTAVYYCARGGMGNYFDYWGQGTLVTVSS |
| 64 | 20B3 VL | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPLTFGGGTKVEIK |
| 65 | 14A2 VH CDR1 | NYALH |
| 66 | 14A2 VH CDR2 | LISYDGSRKHYADSVKG |
| 67 | 14A2 VH CDR3 | LTMVREGG |
| 68 | 14A2 VL1 CDR1 | RASQSVSSSYLA |
| 69 | 14A2 VL1 CDR2 | GASSRAT |
| 70 | 14A2 VL1 CDR3 | QQYGSSPFT |
| 71 | 14A2 VL2 CDR1 | RVSQGISSYLN |
| 72 | 14A2 VL2 CDR2 | SASNLQS |
| 73 | 14A2 VL2 CDR3 | QRTYNAPYT |
| 74 | 14A2 VH | QVQLVESGGGVVQPGRSLRLSCAASGFTFSNYALHWVRQAPGK |
| | | GLEWVALISYDGSRKHYADSVKGRFSISRDNSKNTLYLQMNSL |
| | | RAEDTAVYYCASLTMVREGGQGTLVTVSS |
| 75 | 14A2 VL1 | EIVLTQSPGTLSLSPGERATLSCRASQSVSSSYLAWYQQKPGQ |
| | | APRLLIYGASSRATGIPDRFSGSGSGTDFTLTISRLEPEDFAV |
| | | YYCQQYGSSPFTFGPGTKVDIK |
| 76 | 14A2 VL2 | DIQLTQSPSSLSASVGDRVTITCRVSQGISSYLNWYRQKPGKV |
| | | PKLLIYSASNLQSGVPSRFSGSGSGTDFTLTISSLQPEDVATY |
| | | YGQRTYNAPYTFGGGTKVEIK |
| 77 | 20C1 VH CDR1 | SYAMY |
| 78 | 20C1 VH CDR2 | AIDTDGGTFYADSVRG |
| 79 | 20C1 VH CDR3 | LGEGYFFDY |
| 80 | 20C1 VL CDR1 | RASQSVSSYLA |
| 81 | 20C1 VL CDR2 | DASNRAT |
| 82 | 20C1 VL CDR3 | QQRSNWPPT |
| 83 | 20C1 VH | EAQLVQSGGGLVHPGGSLRLSCADSGFTFSSYAMYWVRQAPGK |
| | | GLEWVSAIDTDGGTFYADSVRGRFTISRDNAKNSLYLQMNGLR |
| | | AEDMAVYFCARLGEGYFFDYWGQGTLVTVSS |
| 84 | 20C1 and | EIVLTQSPATLSLSPGERATLSCRASQSVSSYLAWYQQKPGQA |
| | OX40.21 VL | PRLLIYDASNRATGIPARFSGSGSGTDFTLTISSLEPEDFAVY |
| | | YCQQRSNWPPTFGGGTKVEIK |
| 85 | OX40.21 VH | AIDTDAGTFYADSVRG |
| | CDR2 | |
| 86 | OX40.21 VH | EVQLVQSGGGLVQPGGSLRLSCAGSGFTFSSYAMYWVRQAPGK |
| | | GLEWVSAIDTDAGTFYADSVRGRFTISRDNAKNSLYLQMNSLR |
| | | AEDTAVYFCARLGEGYFFDYWGQGTLVTVSS |
| 87 | human OX40 | DVVSSKPCKPCTWCNLR |
| | epitope | |
| 88 | human OX40 | DSYKPGVDCAPCPPGHFSPGDNQACKPWTNCTLAGK |
| | epitope | |
| 89 | human OX40 | SQNTVCRPCGPGFYNDVVSSKPCKPCTWCNLR |
| | epitope | |
| 90 | human OX40 | PCKPCTWCNLR |
| | epitope | |
| 91 | human OX40 | QLCTATQDTVCR |
| | epitope | |
| 92 | human OX40 | SQNTVCRPCGPGFYN |
| | epitope | |
| 93 | Extra- | LHCVGDTYPSNDRCCHECRPGNGMVSRCSRSQNTVCRPCGPGF |
| | cellular | YNDVVSSKPCKPCTWCNLRSGSERKQLCTATQDTVCRCRAGTQ |
| | domain of | PLDSYKPGVDCAPCPPGHFSPGDNQACKPWTNCTLAGKHTLQP |
| | mature human | ASNSSDAICEDRDPPATQPQETQGPPARPITVQPTEAWPRTSQ |
| | OX40 | GPSTRPVEVPGGRAVAA |
| 94 | OX40.6 heavy | EVQLVQSGGGLVQPGGSLRLSCAGSGFTFSNYAMYWVRQAPGK |
| | chain | GLEWVSAIGIGGDTFYTDSVKGRFTISRDNAKNSLSLQMNSLR |
| | | AEDTAVYYCARMGTGYFFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 95 | OX40.6 light | EIVLTQSPATLSLSPGERATLSCRASQSVSSYLAWYQQKPGQA |
| | chain | PRLLIYDASNRATGIPARFSGSGSGTDFTLTISSLEPEDFAVY |
| | | YCQQRSNWPLTFGPGTKVDIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| | | EVQLVQSGGGLVHPGGSLRLSCAGSGFTFSNYAMYWVRQAPGK |
| | | GLEWVSAIGIGGDTFYTDSVKGRFTISRDNAKNSLSLQMNSLR |
| | | AEDTAVYYCARYGTGYFFDYWGQGTLVTVSSASTKGPSVFPLA |
| | | PSSKSTSGGTAALGCLVKDYFPEPVTVSWNSGALTSGVHTFPA |
| 96 | OX40.7 heavy | VLQSSGLYSLSSVVTVPSSSLGTQTYICNVNHKPSNTKVDKRV |
| | chain | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 97 | OX40.7 light | EIVLTQSPATLSLSPGERATLSCRASQSVSSYLAWYQQKPGQA |
| | chain | PRLLIYDASNRATGIPARFSGSGSGTDFTLTISSLEPEDFAVY |
| | | YCQQRSNWPLTFGPGTKVDIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 98 | OX40.8 heavy | QVQLVESGGGVVQPGRSLRLSCAASGFTFSNYALHWVRQAPGK |
| | chain | GLEWVALISYDGSRKHYADSVKGRFSISRDNSKNTLYLQMNSL |
| | | RAEDTAVYYCASLTMVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 99 | OX40.8 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 100 | OX40.9 heavy | QVQLVESGGGVVQPGRSLRLSCAASGFTFSNYALHWVRQAPGK |
| | chain | GLEWVALISYDGSRKHYADSVKGRFSISRDNSKNTLYLQMNSL |
| | | RAEDTAVYYCASLTYVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 101 | OX40.9 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 102 | OX40.10 heavy | QVQLVESGGGVVQPGRSLRLSCAASGFTFSNYALHWVRQAPGK |
| | chain | GLEWVALISYSGSRKHYADSVKGRFSISRDNSKNTLYLQMNSL |
| | | RAEDTAVYYCASLTMVREGGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 103 | OX40.10 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 104 | OX40.11 heavy | QVQLVESGGGVVQPGRSLRLSCAASGFTFSNYALHWVRQAPGK |
| | chain | GLEWVALISYDSSRKHYADSVKGRFSISRDNSKNTLYLQMNSL |
| | | RAEDTAVYYCASLTMVREGGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 105 | OX40.11 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 106 | OX40.12 heavy | QVQLVESGGGVVQPGRSLRLSCAASGFTFSNYALHWVRQAPGK |
| | chain | GLEWVALISYSGSRKHYADSVKGRFSISRDNSKNTLYLQMNSL |
| | | RAEDTAVYYCASLTMVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 107 | OX40.12 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 108 | OX40.13 heavy | QVQLVESGGGVVQPGRSLRLSCAASGFTFSNYALHWVRQAPGK |
| | chain | GLEWVALISYDSSRKHYADSVKGRFSISRDNSKNTLYLQMNSL |
| | | RAEDTAVYYCASLTMVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 109 | OX40.13 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 110 | OX40.14 heavy | QVQLVESGGGVVQPGRSLRLSCAASGFTFSNYALHWVRQAPGK |
| | chain | GLEWVALISYSGSRKHYADSVKGRFSISRDNSKNTLYLQMNSL |
| | | RAEDTAVYYCASLTYVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 111 | OX40.14 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 112 | OX40.15 heavy | QVQLVESGGGVVQPGRSLRLSCAASGFTFSNYALHWVRQAPGK |
| | chain | GLEWVALISYDSSRKHYADSVKGRFSISRDNSKNTLYLQMNSL |
| | | RAEDTAVYYCASLTYVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 113 | OX40.15 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 114 | OX40.16 heavy | EVQLVQSGGGLVQPGGSLRLSCAGSGFTFSSYAMYWVRQAPGK |
| | chain | GLEWVSAIDTDGGTFYADSVRGRFTISRDNAKNSLYLQMNSLR |
| | | AEDTAVYFCARLGEGYFFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 115 | OX40.16 light | EIVLTQSPATLSLSPGERATLSCRASQSVSSYLAWYQQKPGQA |
| | chain | PRLLIYDASNRATGIPARFSGSGSGTDFTLTISSLEPEDFAVY |
| | (shared by | YCQQRSNWPPTFGGGTKVEIKRTVAAPSVFIFPPSDEQLKSGT |
| | OX40.20, | ASVVCLLNNFYPREAKVQWKVDNALQSGNSQESVTEQDSKDST |
| | OX40.21, | YSLSSTLTLSKADYEKHKVYACEVTHQGLSSPVTKSFNRGEC |
| | OX40.22) | |
| 116 | OX40.17 heavy | EVQLVESGGGLVQPGGSLRLSCAASGFTFSSYDMHWVRQTTGK |
| | chain | GLEWVSVIGTAGDTYYPGSVKGRFTISRENAKNSLYLQMNSLR |
| | | AGDTAVYYCARGGMGNYFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 117 | OX40.17 light | EIVLTQSPATLSLSPGERATLSCRASQSVSSYLAWYQQKPGQA |
| | chain | PRLLIYDASNRATGIPARFSGSGSGTDFTLTISSLEPEDFAVY |
| | | YCQQRSNWPLTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 118 | OX40.18 heavy | QVQLVQSGAEVKKPGASVKVSCKASGYTFTSYDVNWVRQATGQ |
| | chain | GLEWMGWMNPNSGNTGYAPKFQGRVTMTRDTSISTAYMELSSL |
| | | RSEDTAVYYCARIYSSSYNWFDYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPG |
| 119 | OX40.18 light | EIVLTQSPATLSLSPGERATLSCRASQSVSSYLAWYQQKPGQA |
| | chain | PRLLIYDASNRATGIPARFSGSGSGTDFTLTISSLEPEDFAVY |
| | | YCQQRSNWPLTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 120 | OX40.19 heavy | QVQLVESGGGVVQPGRSLRLSCAASGFTFSNYALHWVRQAPGK |
| | chain | GLEWVALISYDGSRKHYADSVKGRFSISRDNSKNTLYLQMNSL |
| | | RAEDTAVYYCASLTLVREWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKRVEP |
| | | KSCDKTHTCPPCPAPELLGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPG |
| 121 | OX40.19 light | EIVLTQSPGTLSLSPGERATLSCRASQSVSSSYLAWYQQKPGQ |
| | chain | APRLLIYGASSRATGIPDRFSGSGSGTDFTLTISRLEPEDFAV |
| | | YYCQQYGSSPFTFGPGTKVDIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 122 | OX40.20 heavy | EVQLVQSGGGLVQPGGSLRLSCAGSGFTFSSYAMYWVRQAPGK |
| | chain | GLEWVSAIDTSGGTFYADSVRGRFTISRDNAKNSLYLQMNSLR |
| | | AEDTAVYFCARLGEGYFFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 123 | OX40.21 heavy | EVQLVQSGGGLVQPGGSLRLSCAGSGFTFSSYAMYWVRQAPGK |
| | chain | GLEWVSAIDTDAGTFYADSVRGRFTISRDNAKNSLYLQMNSLR |
| | | AEDTAVYFCARLGEGYFFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 124 | OX40.22 heavy | EVQLVQSGGGLVQPGGSLRLSCAGSGFTFSSYAMYWVRQAPGK |
| | chain | GLEWVSAIDTSTGTFYADSVRGRFTISRDNAKNSLYLQMNSLR |
| | | AEDTAVYFCARLGEGYFFDYWGQGTLVTVSSASTKGPSVFPLA |
| | | PSSKSTSGGTAALGCLVKDYFPEPVTVSWNSGALTSGVHTFPA |
| | | VLQSSGLYSLSSVVTVPSSSLGTQTYICNVNHKPSNTKVDKRV |
| | | EPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |

What is claimed is:

1. A method of treating a cancer or a solid tumor in a subject in need thereof comprising administering a therapeutically effective amount of:
(a) an OX40 agonist;
(b) a PD-1 pathway inhibitor; and
(c) a CTLA-4 inhibitor to said subject.

2. The method of claim 1, wherein the administering inhibits and/or reduces a rate of tumor growth in the subject.

3. The method of claim 2, wherein the tumor volume is decreased by at least 5%, at least 10%, at least 20%, at least 30%, at least 40%, at least 50%, at least 60%, at least 70%, at least 80%, at least 90%, or 100% compared to a reference (e.g., corresponding tumor volume in a subject who did not receive a therapeutically effective amount of the OX40 agonist, the PD-1 pathway inhibitor, and the CTLA-4 inhibitor).

4. The method of claim 3, wherein the tumor volume is decreased by about 68% compared to a reference (e.g., corresponding tumor volume in a subject who received a dual therapy of anti-CTLA-4 antibody and anti-PD-1 antibody).

5. The method of claim 3, wherein the tumor volume is decreased by about 73% compared to a reference (e.g., corresponding tumor volume in a subject who received a dual therapy of anti-CTLA-4 antibody and anti-PD-1 antibody).

6. The method of claim 3, wherein the tumor volume is decreased by about 30% compared to a reference (e.g., corresponding tumor volume in a subject who received a dual therapy of anti-CTLA-4 antibody and anti-PD-1 antibody).

7. The method of claim 2, wherein the tumor weight is decreased by at least 5%, at least 10%, at least 20%, at least 30%, at least 40%, at least 50%, at least 60%, at least 70%, at least 80%, at least 90%, or at least 100% compared to a reference (e.g., corresponding tumor weight in a subject who did not receive a therapeutically effective amount of the OX40 agonist, the PD-1 pathway inhibitor, and the CTLA-4 inhibitor).

8. The method of claim 7, wherein the tumor weight is decreased by about 40% compared to a reference (e.g., corresponding tumor volume in a subject who received a dual therapy of anti-CTLA-4 antibody and anti-PD-1 antibody).

9. The method of any one of claims 1 to 8, wherein the administering increases the frequency of effector CD4⁺ T cells in a tumor in the subject.

10. The method of any one of claims 1 to 9, wherein the administering increases the frequency of effector CD4⁺ T cells in a peripheral blood in the subject.

11. The method of claim 9 or 10, wherein the frequency of effector CD4⁺ T cells in the tumor and/or in the peripheral blood is increased by at least 5%, at least 10%, at least 20%, at least 30%, at least 40%, at least 50%, at least 60%, at least 70%, at least 80%, at least 90%, or at least 100% compared to a reference.

12. The method of claim 11, wherein the frequency of effector CD4⁺ T cells in the tumor is increased by about 100% compared to a reference (e.g., corresponding value in a subject who received a dual therapy of anti-CTLA-4 antibody and anti-PD-1 antibody).

13. The method of claim 11, wherein the frequency of effector CD4⁺ T cells in the peripheral blood is increased by about 70% compared to a reference (e.g., corresponding value in a subject who received a dual therapy of anti-CTLA-4 antibody and anti-PD-1 antibody).

14. The method of any one of claims 9 to 13, wherein the effector CD4⁺ T cells are Foxp3⁻ Ki67⁺.

15. The method of any one of claims 1 to 14, wherein the administering increases the frequency of effector CD8⁺ T cells in peripheral blood in the subject.

16. The method of claim 15, wherein the frequency of effector CD8⁺ T cells in the peripheral blood is increased by at least 5%, at least 10%, at least 20%, at least 30%, at least 40%, at least 50%, at least 60%, at least 70%, at least 80%, at least 90%, or at least 100% compared to a reference.

17. The method of claim 15, wherein the frequency of effector CD8⁺ T cells in the peripheral blood is increased by about 33% compared to a reference (e.g., corresponding value in a subject who received a dual therapy of anti-CTLA-4 antibody and anti-PD-1 antibody).

18. The method of any one of claims 15 to 17, wherein the effector CD8⁺ T cells are PDF Ki67⁺.

19. The method of any one of claims 1 to 18, wherein the administering reduces the frequency of regulatory CD4⁺ T cells in a tumor in the subject.

20. The method of claim 19, wherein the frequency of regulatory CD4⁺ T cells in the tumor is reduced by at least 5%, at least 10%, at least 20%, at least 30%, at least 40%, at least 50%, at least 60%, at least 70%, at least 80%, at least 90%, or 100% compared to a reference.

21. The method of claim 20, wherein the frequency of regulatory T cells in the tumor is reduced by about 30% compared to a reference (e.g., corresponding value in a subject who received a dual therapy of anti-CTLA-4 antibody and anti-PD-1 antibody).

22. The method of any one of claims 19 to 21, wherein the regulatory CD4⁺ T cells are Foxp3⁺.

23. The method of any one of claims 1 to 22, wherein the administering reduces the frequency of exhausted T cells in a tumor in the subject.

24. The method of claim 23, wherein the frequency of exhausted T cells in the tumor is reduced by at least 5%, at least 10%, at least 20%, at least 30%, at least 40%, at least 50%, at least 60%, at least 70%, at least 80%, at least 90%, or 100% compared to a reference.

25. The method of claim 24, wherein the frequency of exhausted T cells in the tumor by about 71% compared to a reference (e.g., corresponding value in a subject who received a dual therapy of anti-CTLA-4 antibody and anti-PD-1 antibody).

26. The method of any one of claims 1 to 25, wherein the administering reduces the frequency of granulocytic myeloid-derived suppressor cells (gMDSC) in a peripheral blood in the subject.

27. The method of claim 26, wherein the frequency of gMDSC in the peripheral blood is reduced by at least 5%, at least 10%, at least 20%, at least 30%, at least 40%, at least 50%, at least 60%, at least 70%, at least 80%, at least 90%, or 100% compared to a reference.

28. The method of claim 27, wherein the frequency of gMDSC in the peripheral blood is reduced by about 30% compared to a reference (e.g., corresponding value in a subject who received a dual therapy of anti-CTLA-4 antibody and anti-PD-1 antibody).

29. The method of any one of claims 1 to 28, wherein the administering increases a mean fluorescence intensity (MFI) of PD-L1 expression on a gMDSC in a tumor in the subject.

30. The method of claim 29, wherein the mean fluorescence intensity (MFI) of PD-L1 expression is increased by at least 5%, at least 10%, at least 20%, at least 30%, at least 40%, at least 50%, at least 60%, at least 70%, at least 80%, at least 90%, or at least 100% compared to a reference.

31. The method of claim 30, wherein the MFI of PD-L1 expression is increased by about 89% compared to a reference (e.g., corresponding value in a subject who received a dual therapy of anti-CTLA-4 antibody and anti-PD-1 antibody).

32. The method of any one of claims 1 to 31, wherein the administering reduces a mean fluorescence intensity (MFI) of CCR2 expression on a monocyte in a peripheral blood in the subject.

33. The method of claim 32, wherein the mean fluorescence intensity (MFI) of CCR2 expression on the gMDSC is reduced increased by at least 5%, at least 10%, at least 20%, at least 30%, at least 40%, at least 50%, at least 60%, at least 70%, at least 80%, at least 90%, or at least 100% compared to a reference.

34. The method of claim 33, wherein the MFI of CCR2 expression is reduced by about 54% compared to a reference (e.g., corresponding value in a subject who received a dual therapy of anti-CTLA-4 antibody and anti-PD-1 antibody).

35. The method of any one of claims 26 to 33, wherein the gMDSC is Ly6G⁺⁺.

36. The method of any one of claims 1 to 35, wherein the administering increases a level of a proinflammatory cytokine in a tumor in the subject.

37. The method of claim 36, wherein the level of the proinflammatory cytokine in the tumor is increased by at least 5%, at least 10%, at least 20%, at least 30%, at least 40%, at least 50%, at least 60%, at least 70%, at least 80%, at least 90%, or at least 100% compared to a reference.

38. The method of claim 36 or 37, wherein the proinflammatory cytokine comprises IFN-γ.

39. The method of claim 38, wherein the level of IFN-y is increased by about 39% compared to a reference (e.g., corresponding value in a subject who received a dual therapy of anti-CTLA-4 antibody and anti-PD-1 antibody).

40. The method of any one of claims 1 to 39, wherein the administering increases the frequency of classical monocytes in the peripheral blood in the subject.

41. The method of claim 40, wherein the frequency of classical monocytes is increased by at least 5%, at least 10%, at least 20%, at least 30%, at least 40%, at least 50%, at least 60%, at least 70%, at least 80%, at least 90%, or at least 100% compared to a reference (e.g., corresponding value in a subject who received a dual therapy of anti-CTLA-4 antibody and anti-PD-1 antibody).

42. The method of claim 41, wherein the frequency of classical monocytes is increased by about 80% compared to a reference (e.g., corresponding value in a subject who received a dual therapy of anti-CTLA-4 antibody and anti-PD-1 antibody).

43. The method of any one of claims 40 to 42, wherein the classical monocytes are Ly6C⁺⁺.

44. The method of any one of claims 3 to 43, wherein the reference is a cancer subject who did not receive a therapeutically effective amount of the OX40 agonist, the CTLA-4 inhibitor, and the PD-1 inhibitor.

45. The method of any one of claims 3 to 44, wherein the reference is a cancer subject who received an anti-PD-1 antibody monotherapy, an anti-CTLA-4 antibody monotherapy, or a dual combination of an anti-PD-1 antibody and an anti-CTLA-4 antibody.

46. The method of any one of claims 1 to 45, wherein the OX40 agonist, the CTLA-4 inhibitor, and the PD-1 inhibitor are administered to the subject concurrently.

47. The method of any one of claims 1 to 45, wherein the OX40 agonist, the CTLA-4 inhibitor, and the PD-1 inhibitor are administered to the subject sequentially.

48. The method of any one of claims 1 to 45, wherein the OX40 agonist, the CTLA-4 inhibitor, and the PD-1 inhibitor are administered to the subject in any order.

49. The method of any one of claims 1 to 45, wherein the OX40 agonist is an anti-OX40 antibody.

50. The method of claim 49, wherein the anti-OX40 antibody comprises a heavy chain CDR1, CDR2, and CDR3, and a light chain CDR1, CDR2, and CDR3, wherein
(a) the heavy chain CDR1 comprises an amino acid sequence set forth as SEQ ID NO: 1, 9, 21, 29, 41, 49, 57, 65, or 77;
(b) the heavy chain CDR2 comprises an amino acid sequence set forth as SEQ ID NO: 2, 10, 22, 30, 42, 50, 58, 66, 78, or 85;
(c) the heavy chain CDR3 comprises an amino acid sequence set forth as SEQ ID NO: 3, 11, 23, 31, 43, 51, 59, 67, or 79;
(d) the light chain CDR1 comprises an amino acid sequence set forth as SEQ ID NO: 4, 12, 15, 24, 32, 35, 44, 52, 60, 68, 71, or 80;
(e) the light chain CDR2 comprises an amino acid sequence set forth as SEQ ID NO: 5, 13, 16, 25, 33, 36, 45, 53, 61, 69, 72, or 81; and
(f) the light chain CDR3 comprises an amino acid sequence set forth as SEQ ID NO: 6, 14, 17, 26, 34, 37, 46, 54, 62, 70, 73, or 82.

51. The method of claim 49 or 50, wherein the anti-OX40 antibody comprises a heavy chain variable region (VH) and a light chain variable region (VL), wherein the VH comprises an amino acid sequence set forth as SEQ ID NO: 7, 18, 27, 38, 47, 55, 63, 74, 83, or 86, and wherein the VL comprises an amino acid sequence set forth as SEQ ID NO: 8, 19, 20, 28, 39, 40, 48, 56, 64, 75, 76, or 84.

52. The method of any one of claims 49 to 51, wherein the anti-OX40 antibody comprises a VH and a VL, wherein the VH comprises the CDR1, CDR2, and CDR3 sequences set forth as SEQ ID NOs: 77, 85, and 79, respectively, and wherein the VL comprises the CDR1, CDR2, and CDR3 sequences set forth as SEQ ID NOs: 80, 81, and 82, respectively.

53. The method of claim 49, wherein the anti-OX40 antibody is tavolixizumab (MEDI-0562), pogalizumab (MOXR0916, RG7888), GSK3174998, ATOR-1015, MEDI-6383, MEDI-6469, BMS986178, PF-04518600, RG7888 (MOXR0916), INCAGN1949, KHK4083, ENUM004, or ABBV-368.

54. The method of any one of claims 1 to 53, wherein the CTLA-4 inhibitor is an anti-CTLA-4 antibody.

55. The method of claim 54, wherein the anti-CTLA-4 antibody is ipilimumab (YERVOY®), tremelimumab (ticilimumab; CP-675,206), AGEN-1884, or ATOR-1015.

56. The method of any one of claims 1 to 55, wherein the PD-1 inhibitor is an anti-PD-1 antibody.

57. The method of claim 56, wherein the anti-PD-1 antibody is nivolumab (OPDIVO®), pembrolizumab (KEYTRUDA®P; MK-3475), pidilizumab (CT-011), PDR001, MEDI0680 (AMP-514), TSR-042, REGN2810, JS001, PF-06801591, BGB-A317, BI 754091, or SHR-1210.

58. The method of any one of claims 1 to 55, wherein the PD-1 inhibitor is an anti-PD-L1 antibody.

59. The method of claim 58, wherein the anti-PD-L1 antibody is atezolizumab (TECENTRIQ®; RG7446; MPDL3280A; R05541267), durvalumab (MEDI4736), BMS-936559, avelumab (BAVENCIO®), LY3300054, CX-072 (Proclaim-CX-072), FAZ053, KNO35, or MDX-1105.

60. The method of any one of claims 1 to 55, wherein the PD-1 inhibitor is an anti-PD-L2 antibody.

61. The method of claim 60, wherein the anti-PD-L2 antibody is rHIgM12B7.

62. The method of any one of claims 1 to 61, wherein the cancer is selected from the group consisting of a liver cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, cancers of the childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combination thereof.

63. The method of any one of claims 1 to 62, wherein the cancer is melanoma (e.g., metastatic malignant melanoma), renal cancer (e.g. clear cell carcinoma), prostate cancer (e.g. hormone refractory prostate adenocarcinoma), breast cancer, colon cancer, lung cancer (e.g. non-small cell lung cancer), or any combination thereof

64. The method of claim 63, wherein the cancer is breast cancer.

65. The method of claim 64, wherein the breast cancer is triple negative breast cancer.

66. The method of any one of claims 1 to 65 wherein the cancer is refractory to a first line treatment.

67. The method of any one of claims 1 to 66, wherein the cancer is refractory to a monotherapy comprising an OX40 inhibitor, a CTLA-4 inhibitor, or a PD-1 inhibitor.

68. The method of any one of claims 1 to 66, wherein the cancer is refractory to a doublet combination therapy comprising an OX40 inhibitor, a CTLA-4 inhibitor, or a PD-1 inhibitor.

69. The method of any one of claims 1 to 68, further comprising administering at least one additional therapeutic agent.

70. A method of expanding effector CD4⁺ T cell populations in a human patient comprising administering a therapeutically effective amount of:
(a) an OX-40 agonist;
(b) a PD-1 pathway inhibitor; and
(c) a CTLA-4 inhibitor to said patient.

71. The method of claim 70, wherein the effector CD4⁺ T cells are CD4⁺ Foxp3⁻.

72. The method of claims 70 and 71, wherein the expansion occurs in both malignant tumors and blood.

73. A method of reducing exhausted effector CD4⁺ T cells in a human patient comprising administering a therapeutically effective amount of:
(a) an OX-40 agonist;
(b) a PD-1 pathway inhibitor; and
(c) a CTLA-4 inhibitor to said patient.

74. A method of reducing the presence of granulocytic myeloid-derived suppressor cells in a human patient comprising administering a therapeutically effective amount of:
(a) an OX-40 agonist;
(b) a PD-1 pathway inhibitor; and
(c) a CTLA-4 inhibitor to said patient.

75. A method of increasing proinflammatory cytokine production by tumors in a human patient comprising administering a therapeutically effective amount of:
(a) an OX-40 agonist;
(b) a PD-1 pathway inhibitor; and
(c) a CTLA-4 inhibitor to said patient.

76. A method of treating a triple negative breast cancer in a subject in need thereof comprises administering to the subject a therapeutically effective amount of: (i) BMS986178, (ii) nivolumab (OPDIVO®), and (iii) ipilimumab (YERVOY®).

77. A method of treating a triple negative breast cancer in a subject in need thereof comprises administering to the subject a therapeutically effective amount of: (i) tavolixizumab (MEDI-0562), (ii) durvalumab (IMIFINZI®; MEDI4736), and (iii) tremelimumab (ticilimumab and CP-675,206).

78. A method of treating a treating a triple negative breast cancer in a subject in need thereof comprises administering to the subject a therapeutically effective amount of: (i) pogalizumab (MOXR0916, RG7888), (ii) atezolizumab (TECENTRIQ®, RG7446), and (iii) ipilimumab (YERVOY®).

79. A method of determining the efficacy of a cancer treatment in a subject, comprising measuring a frequency of effector CD4⁺ T cells (e.g., Foxp3⁻, Ki67⁺) in a peripheral blood of the subject, wherein an increase in the frequency of effector CD4⁺ T cells compared to a reference value (e.g., corresponding value in the subject prior to treatment) indicates that the cancer treatment is efficacious.

80. A method of determining the efficacy of a cancer treatment in a subject, comprising measuring a frequency of effector CD8⁺ T cells (e.g., PDF, Ki67⁺) in a peripheral blood of the subject, wherein an increase in the frequency of effector CD8⁺ T cells compared to a reference value (e.g., corresponding value in the subject prior to treatment) indicates that the cancer treatment is efficacious.

81. A method of determining the efficacy of a cancer treatment in a subject, comprising measuring a frequency of regulatory T cells (e.g., Foxp3⁺ CD4⁺ T cells) in a peripheral blood of the subject, wherein an increase in the frequency of effector CD4⁺ T cells compared to a reference value (e.g., corresponding value in the subject prior to treatment) indicates that the cancer treatment is efficacious.

82. The method of any one of claims 79 to 81, wherein the cancer treatment comprises administering to the subject a therapeutically effective amount of : (a) an OX40 agonist; (b) a PD-1 pathway inhibitor; and (c) a CTLA-4 inhibitor to the subject.


SEQUENCE LISTING (124 sequences)

SEQ ID NO: 1 - protein, 5 aa, Artificial Sequence
  3F4 VH CDR1
SYDVN

SEQ ID NO: 2 - protein, 17 aa, Artificial Sequence
  3F4 VH CDR2
WMNPNSGNTGYAPKFQG

SEQ ID NO: 3 - protein, 11 aa, Artificial Sequence
  3F4 VH CDR3
IYSSSYNWFDP

SEQ ID NO: 4 - protein, 11 aa, Artificial Sequence
  3F4 VL CDR1
RASQSVSSYLA

SEQ ID NO: 5 - protein, 7 aa, Artificial Sequence
  3F4 VL CDR2
DASNRAT

SEQ ID NO: 6 - protein, 9 aa, Artificial Sequence
  3F4 VL CDR3
QQRSNWPLT

SEQ ID NO: 7 - protein, 120 aa, Artificial Sequence
  3F4 VH
QVQLVQSGAEVKKPGASVKVSCKASGNTFTSYDVNWVRQATGQGLEWMGWMNPNSGNTGYAPKFQGRVTMTRNTSISTAYMELSSLRSEDTAVYYCARIYSSSYNWFDPWGQGTLVTVSS

SEQ ID NO: 8 - protein, 107 aa, Artificial Sequence
  3F4 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 9 - protein, 5 aa, Artificial Sequence
  14B6 VH CDR1
SNWIG

SEQ ID NO: 10 - protein, 17 aa, Artificial Sequence
  14B6 VH CDR2
FIYPGDSDTRYSPSFQG

SEQ ID NO: 11 - protein, 9 aa, Artificial Sequence
  14B6 VH CDR3
YGDDWYFDL

SEQ ID NO: 12 - protein, 11 aa, Artificial Sequence
  14B6 VL1 CDR1
RASQSVSSYLA

SEQ ID NO: 13 - protein, 7 aa, Artificial Sequence
  14B6 VL1 CDR2
DASNRAT

SEQ ID NO: 14 - protein, 9 aa, Artificial Sequence
  14B6 VL1 CDR3
QQRGDWPIT

SEQ ID NO: 15 - protein, 11 aa, Artificial Sequence
  14B6 VL2 CDR1
RASQGISSWLA

SEQ ID NO: 16 - protein, 7 aa, Artificial Sequence
  14B6 VL2 CDR2
AASSLQS

SEQ ID NO: 17 - protein, 10 aa, Artificial Sequence
  14B6 VL2 CDR3
QQYNSYPRIT

SEQ ID NO: 18 - protein, 118 aa, Artificial Sequence
  14B6 VH
EVQLEQSGAEVKKPGESLKISCKGSGYSFTSNWIGWVRQMPGKGLEWMGFIYPGDSDTRYSPSFQGQVTISADKSISTAYLQWSSLKASDIAMYYCARYGDDWYFDLWGRGTLVTVSS

SEQ ID NO: 19 - protein, 107 aa, Artificial Sequence
  14B6 VL1
EIVLTQSPATLSLSPGERATLSCRASQSVSSYLAWFQQRPGQAPRLLIYDASNRATGIPARFSGSGSGTDFSLTISSLEPEDFAVYYCQQRGDWPITFGQGTRLEIK

SEQ ID NO: 20 - protein, 108 aa, Artificial Sequence
  14B6 VL2
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRITFGQGTRLEIK

SEQ ID NO: 21 - protein, 5 aa, Artificial Sequence
  23H3 VH CDR1
NYAMY

SEQ ID NO: 22 - protein, 16 aa, Artificial Sequence
  23H3 VH CDR2
AIGIGGDTFYTDSVKG

SEQ ID NO: 23 - protein, 9 aa, Artificial Sequence
  23H3 VH CDR3
MGTGYFFDY

SEQ ID NO: 24 - protein, 11 aa, Artificial Sequence
  23H3 VL CDR1
RASQSVSSYLA

SEQ ID NO: 25 - protein, 7 aa, Artificial Sequence
  23H3 VL CDR2
DASNRAT

SEQ ID NO: 26 - protein, 9 aa, Artificial Sequence
  23H3 VL CDR3
QQRSNWPLT

SEQ ID NO: 27 - protein, 117 aa, Artificial Sequence
  23H3 VH
EVQLVQSGGGLVHPGGSLRLSCAGSGFTFSNYAMYWVRQAPGKGLEWVSAIGIGGDTFYTDSVKGRFTISRDNAKNSLSLQMNSLRAEDMAVYYCARMGTGYFFDYWGQGTLVTVSS

SEQ ID NO: 28 - protein, 107 aa, Artificial Sequence
  23H3 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGPGTKVDIK

SEQ ID NO: 29 - protein, 5 aa, Artificial Sequence
  6E1 VH CDR1
SFAMH

SEQ ID NO: 30 - protein, 17 aa, Artificial Sequence
  6E1 VH CDR2
VISYDGSIKYYTDSVKG

SEQ ID NO: 31 - protein, 12 aa, Artificial Sequence
  6E1 VH CDR3
DGNYGSARYFQH

SEQ ID NO: 32 - protein, 11 aa, Artificial Sequence
  6E1 VL1 CDR1
RASQGISSWLA

SEQ ID NO: 33 - protein, 7 aa, Artificial Sequence
  6E1 VL1 CDR2
AASSLQS

SEQ ID NO: 34 - protein, 9 aa, Artificial Sequence
  6E1 VL1 CDR3
QQYNSYPRT

SEQ ID NO: 35 - protein, 11 aa, Artificial Sequence
  6E1 VL2 CDR1
RASQSVSSYLA

SEQ ID NO: 36 - protein, 7 aa, Artificial Sequence
  6E1 VL2 CDR2
DASNRAT

SEQ ID NO: 37 - protein, 9 aa, Artificial Sequence
  6E1 VL2 CDR3
QQRSNWPYT

SEQ ID NO: 38 - protein, 121 aa, Artificial Sequence
  6E1 VH
QVQLVESGGGVVQPGRSLRLSCAASGFTFSSFAMHWVRQAPGKGLEWVTVISYDGSIKYYTDSVKGRFTFSRDNSKNTLYLQMNSLRAEDTAVYYCTRDGNYGSARYFQHWGQGTLVTVSS

SEQ ID NO: 39 - protein, 107 aa, Artificial Sequence
  6E1 VL1
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 40 - protein, 107 aa, Artificial Sequence
  6E1 VL2
EIVLTQSPATLSLSPGERATLSCRASQSVSSYLAWYQQKPGQAPRLLIYDASNRATGIPARFSGSGSGTDFTLTISSLEPEDFAVYYCQQRSNWPYTFGQGTKLEIK

SEQ ID NO: 41 - protein, 5 aa, Artificial Sequence
  18E9 VH CDR1
SSAMH

SEQ ID NO: 42 - protein, 16 aa, Artificial Sequence
  18E9 VH CDR2
AIGTGGDTYYADSVKG

SEQ ID NO: 43 - protein, 12 aa, Artificial Sequence
  18E9 VH CDR3
DFYDILTGIFDY

SEQ ID NO: 44 - protein, 11 aa, Artificial Sequence
  18E9 VL CDR1
RASQGISSWLA

SEQ ID NO: 45 - protein, 7 aa, Artificial Sequence
  18E9 VL CDR2
AASSLQS

SEQ ID NO: 46 - protein, 9 aa, Artificial Sequence
  18E9 VL CDR3
QQANSFPST

SEQ ID NO: 47 - protein, 120 aa, Artificial Sequence
  18E9 VH
EVQLVQSGGGLVHPGGSLRLSCAHSGFTFTSSAMHWVRQAPGKGLEWISAIGTGGDTYYADSVKGRFTISRDNAKNSLYLQINSLRAEDMAVYYCARDFYDILTGIFDYWGQGTLVTVSS

SEQ ID NO: 48 - protein, 107 aa, Artificial Sequence
  18E9 VL
DIQMTQSPSSVSASVGDRVTITCRASQGISSWLAWYQHKPGKAPKLLIYAASSLQSGVPSRFSGSGSGTDFTLTISSLQPEDFATYYCQQANSFPSTFGQGTKVEIK

SEQ ID NO: 49 - protein, 5 aa, Artificial Sequence
  8B11 VH CDR1
SDAMY

SEQ ID NO: 50 - protein, 16 aa, Artificial Sequence
  8B11 VH CDR2
AIGIGGDTYYTDSVMG

SEQ ID NO: 51 - protein, 9 aa, Artificial Sequence
  8B11 VH CDR3
LGMGYYFDY

SEQ ID NO: 52 - protein, 11 aa, Artificial Sequence
  8B11 VL CDR1
RASQSVSSYLA

SEQ ID NO: 53 - protein, 7 aa, Artificial Sequence
  8B11 VL CDR2
DASNRAT

SEQ ID NO: 54 - protein, 9 aa, Artificial Sequence
  8B11 VL CDR3
QQRSNWPPT

SEQ ID NO: 55 - protein, 136 aa, Artificial Sequence
  8B11 VH
MEFVLSWVFLVAILKGVQCEIQLVQSGGGLVHPGGSLRLSCAGSGFTFSSDAMYWVRQAPGKGLEWVSAIGIGGDTYYTDSVMGRFTISRDNAKNSLYLQMNSLRAEDMAVYYCARLGMGYYFDYWGQGTLVTVSS

SEQ ID NO: 56 - protein, 107 aa, Artificial Sequence [sequence omitted]

SEQ ID NO: 57 - protein, 5 aa, Artificial Sequence
  20B3 VH CDR1
SYDMH

SEQ ID NO: 58 - protein, 16 aa, Artificial Sequence
  20B3 VH CDR2
VIGTAGDTYYPGSVKG

SEQ ID NO: 59 - protein, 9 aa, Artificial Sequence
  20B3 VH CDR3
GGMGNYFDY

SEQ ID NO: 60 - protein, 11 aa, Artificial Sequence
  20B3 VL CDR1
RASQSVSSYLA

SEQ ID NO: 61 - protein, 7 aa, Artificial Sequence
  20B3 VL CDR2
DASNRAT

SEQ ID NO: 62 - protein, 9 aa, Artificial Sequence
  20B3 VL CDR3
QQRSNWPLT

SEQ ID NO: 63 - protein, 117 aa, Artificial Sequence
  20B3 VH
EVQLVESGGGLVQPGGSLRLSCAASGFTFSSYDMHWVRQTTGKGLEWVSVIGTAGDTYYPGSVKGRFTISRENAKNSLYLQMNSLRAGDTAVYYCARGGMGNYFDYWGQGTLVTVSS

SEQ ID NO: 64 - protein, 107 aa, Artificial Sequence; identical to SEQ ID NO: 8
  20B3 VL

SEQ ID NO: 65 - protein, 5 aa, Artificial Sequence
  14A2 VH CDR1
NYALH

SEQ ID NO: 66 - protein, 17 aa, Artificial Sequence
  14A2 VH CDR2
LISYDGSRKHYADSVKG

SEQ ID NO: 67 - protein, 8 aa, Artificial Sequence
  14A2 VH CDR3
LTMVREGG

SEQ ID NO: 68 - protein, 12 aa, Artificial Sequence
  14A2 VL1 CDR1
RASQSVSSSYLA

SEQ ID NO: 69 - protein, 7 aa, Artificial Sequence
  14A2 VL1 CDR2
GASSRAT

SEQ ID NO: 70 - protein, 9 aa, Artificial Sequence
  14A2 VL1 CDR3
QQYGSSPFT

SEQ ID NO: 71 - protein, 11 aa, Artificial Sequence
  14A2 VL2 CDR1
RVSQGISSYLN

SEQ ID NO: 72 - protein, 7 aa, Artificial Sequence
  14A2 VL2 CDR2
SASNLQS

SEQ ID NO: 73 - protein, 9 aa, Artificial Sequence
  14A2 VL2 CDR3
QRTYNAPYT

SEQ ID NO: 74 - protein, 115 aa, Artificial Sequence
  14A2 VH
QVQLVESGGGVVQPGRSLRLSCAASGFTFSNYALHWVRQAPGKGLEWVALISYDGSRKHYADSVKGRFSISRDNSKNTLYLQMNSLRAEDTAVYYCASLTMVREGGQGTLVTVSS

SEQ ID NO: 75 - protein, 108 aa, Artificial Sequence
  14A2 VL1
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 76 - protein, 107 aa, Artificial Sequence
  14A2 VL2
DIQLTQSPSSLSASVGDRVTITCRVSQGISSYLNWYRQKPGKVPKLLIYSASNLQSGVPSRFSGSGSGTDFTLTISSLQPEDVATYYGQRTYNAPYTFGGGTKVEIK

SEQ ID NO: 77 - protein, 5 aa, Artificial Sequence
  20C1 VH CDR1
SYAMY

SEQ ID NO: 78 - protein, 16 aa, Artificial Sequence
  20C1 VH CDR2
AIDTDGGTFYADSVRG

SEQ ID NO: 79 - protein, 9 aa, Artificial Sequence
  20C1 VH CDR3
LGEGYFFDY

SEQ ID NO: 80 - protein, 11 aa, Artificial Sequence
  20C1 VL CDR1
RASQSVSSYLA

SEQ ID NO: 81 - protein, 7 aa, Artificial Sequence
  20C1 VL CDR2
DASNRAT

SEQ ID NO: 82 - protein, 9 aa, Artificial Sequence
  20C1 VL CDR3
QQRSNWPPT

SEQ ID NO: 83 - protein, 117 aa, Artificial Sequence
  20C1 VH
EAQLVQSGGGLVHPGGSLRLSCADSGFTFSSYAMYWVRQAPGKGLEWVSAIDTDGGTFYADSVRGRFTISRDNAKNSLYLQMNGLRAEDMAVYFCARLGEGYFFDYWGQGTLVTVSS

SEQ ID NO: 84 - protein, 107 aa, Artificial Sequence
  20C1 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIK

SEQ ID NO: 85 - protein, 16 aa, Artificial Sequence
  OX40.21 VH CDR2
AIDTDAGTFYADSVRG

SEQ ID NO: 86 - protein, 117 aa, Artificial Sequence
  OX40.21 VH
EVQLVQSGGGLVQPGGSLRLSCAGSGFTFSSYAMYWVRQAPGKGLEWVSAIDTDAGTFYADSVRGRFTISRDNAKNSLYLQMNSLRAEDTAVYFCARLGEGYFFDYWGQGTLVTVSS

SEQ ID NO: 87 - protein, 17 aa, Artificial Sequence
  human OX40 epitope
DVVSSKPCKPCTWCNLR

SEQ ID NO: 88 - protein, 36 aa, Artificial Sequence
  human OX40 epitope
DSYKPGVDCAPCPPGHFSPGDNQACKPWTNCTLAGK

SEQ ID NO: 89 - protein, 32 aa, Artificial Sequence
  human OX40 epitope
SQNTVCRPCGPGFYNDVVSSKPCKPCTWCNLR

SEQ ID NO: 90 - protein, 11 aa, Artificial Sequence
  human OX40 epitope
PCKPCTWCNLR

SEQ ID NO: 91 - protein, 12 aa, Artificial Sequence
  human OX40 epitope
QLCTATQDTVCR

SEQ ID NO: 92 - protein, 15 aa, Artificial Sequence
  human OX40 epitope
SQNTVCRPCGPGFYN

SEQ ID NO: 93 - protein, 189 aa, Artificial Sequence
  Extracellular domain of mature human OX40
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAA

SEQ ID NO: 94 - protein, 446 aa, Artificial Sequence
  OX40.6 heavy chain
EVQLVQSGGGLVQPGGSLRLSCAGSGFTFSNYAMYWVRQAPGKGLEWVSAIGIGGDTFYTDSVKGRFTISRDNAKNSLSLQMNSLRAEDTAVYYCARMGTGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 214 aa, Artificial Sequence
  OX40.6 light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 446 aa, Artificial Sequence
  OX40.7 heavy chain
EVQLVQSGGGLVHPGGSLRLSCAGSGFTFSNYAMYWVRQAPGKGLEWVSAIGIGGDTFYTDSVKGRFTISRDNAKNSLSLQMNSLRAEDTAVYYCARYGTGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 214 aa, Artificial Sequence; identical to SEQ ID NO: 95
  OX40.7 light chain

SEQ ID NO: 98 - protein, 444 aa, Artificial Sequence
  OX40.8 heavy chain
QVQLVESGGGVVQPGRSLRLSCAASGFTFSNYALHWVRQAPGKGLEWVALISYDGSRKHYADSVKGRFSISRDNSKNTLYLQMNSLRAEDTAVYYCASLTMVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 215 aa, Artificial Sequence
  OX40.8 light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 444 aa, Artificial Sequence
  OX40.9 heavy chain
QVQLVESGGGVVQPGRSLRLSCAASGFTFSNYALHWVRQAPGKGLEWVALISYDGSRKHYADSVKGRFSISRDNSKNTLYLQMNSLRAEDTAVYYCASLTYVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 215 aa, Artificial Sequence; identical to SEQ ID NO: 99
  OX40.9 light chain

SEQ ID NO: 102 - protein, 444 aa, Artificial Sequence
  OX40.10 heavy chain
QVQLVESGGGVVQPGRSLRLSCAASGFTFSNYALHWVRQAPGKGLEWVALISYSGSRKHYADSVKGRFSISRDNSKNTLYLQMNSLRAEDTAVYYCASLTMVREGGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 215 aa, Artificial Sequence; identical to SEQ ID NO: 99
  OX40.10 light chain

SEQ ID NO: 104 - protein, 444 aa, Artificial Sequence
  OX40.11 heavy chain
QVQLVESGGGVVQPGRSLRLSCAASGFTFSNYALHWVRQAPGKGLEWVALISYDSSRKHYADSVKGRFSISRDNSKNTLYLQMNSLRAEDTAVYYCASLTMVREGGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 215 aa, Artificial Sequence; identical to SEQ ID NO: 99
  OX40.11 light chain

SEQ ID NO: 106 - protein, 444 aa, Artificial Sequence
  OX40.12 heavy chain
QVQLVESGGGVVQPGRSLRLSCAASGFTFSNYALHWVRQAPGKGLEWVALISYSGSRKHYADSVKGRFSISRDNSKNTLYLQMNSLRAEDTAVYYCASLTMVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 215 aa, Artificial Sequence; identical to SEQ ID NO: 99
  OX40.12 light chain

SEQ ID NO: 108 - protein, 444 aa, Artificial Sequence
  OX40.13 heavy chain
QVQLVESGGGVVQPGRSLRLSCAASGFTFSNYALHWVRQAPGKGLEWVALISYDSSRKHYADSVKGRFSISRDNSKNTLYLQMNSLRAEDTAVYYCASLTMVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 215 aa, Artificial Sequence; identical to SEQ ID NO: 99
  OX40.13 light chain

SEQ ID NO: 110 - protein, 444 aa, Artificial Sequence
  OX40.14 heavy chain
QVQLVESGGGVVQPGRSLRLSCAASGFTFSNYALHWVRQAPGKGLEWVALISYSGSRKHYADSVKGRFSISRDNSKNTLYLQMNSLRAEDTAVYYCASLTYVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 215 aa, Artificial Sequence; identical to SEQ ID NO: 99
  OX40.14 light chain

SEQ ID NO: 112 - protein, 444 aa, Artificial Sequence
  OX40.15 heavy chain
QVQLVESGGGVVQPGRSLRLSCAASGFTFSNYALHWVRQAPGKGLEWVALISYDSSRKHYADSVKGRFSISRDNSKNTLYLQMNSLRAEDTAVYYCASLTYVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 215 aa, Artificial Sequence; identical to SEQ ID NO: 99
  OX40.15 light chain

SEQ ID NO: 114 - protein, 446 aa, Artificial Sequence
  OX40.16 heavy chain
EVQLVQSGGGLVQPGGSLRLSCAGSGFTFSSYAMYWVRQAPGKGLEWVSAIDTDGGTFYADSVRGRFTISRDNAKNSLYLQMNSLRAEDTAVYFCARLGEGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 214 aa, Artificial Sequence
  OX40.16 light chain (shared by OX40.20, OX40.21, OX40.22)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 446 aa, Artificial Sequence
  OX40.17 heavy chain
EVQLVESGGGLVQPGGSLRLSCAASGFTFSSYDMHWVRQTTGKGLEWVSVIGTAGDTYYPGSVKGRFTISRENAKNSLYLQMNSLRAGDTAVYYCARGGMGNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 214 aa, Artificial Sequence
  OX40.17 light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 449 aa, Artificial Sequence
  OX40.18 heavy chain
QVQLVQSGAEVKKPGASVKVSCKASGYTFTSYDVNWVRQATGQGLEWMGWMNPNSGNTGYAPKFQGRVTMTRDTSISTAYMELSSLRSEDTAVYYCARIYSSSYNWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 214 aa, Artificial Sequence; identical to SEQ ID NO: 117
  OX40.18 light chain

SEQ ID NO: 120 - protein, 444 aa, Artificial Sequence
  OX40.19 heavy chain
QVQLVESGGGVVQPGRSLRLSCAASGFTFSNYALHWVRQAPGKGLEWVALISYDGSRKHYADSVKGRFSISRDNSKNTLYLQMNSLRAEDTAVYYCASLTLVREWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 215 aa, Artificial Sequence; identical to SEQ ID NO: 99
  OX40.19 light chain

SEQ ID NO: 122 - protein, 446 aa, Artificial Sequence
  OX40.20 heavy chain
EVQLVQSGGGLVQPGGSLRLSCAGSGFTFSSYAMYWVRQAPGKGLEWVSAIDTSGGTFYADSVRGRFTISRDNAKNSLYLQMNSLRAEDTAVYFCARLGEGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 446 aa, Artificial Sequence
  OX40.21 heavy chain
EVQLVQSGGGLVQPGGSLRLSCAGSGFTFSSYAMYWVRQAPGKGLEWVSAIDTDAGTFYADSVRGRFTISRDNAKNSLYLQMNSLRAEDTAVYFCARLGEGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 446 aa, Artificial Sequence
  OX40.22 heavy chain
EVQLVQSGGGLVQPGGSLRLSCAGSGFTFSSYAMYWVRQAPGKGLEWVSAIDTSTGTFYADSVRGRFTISRDNAKNSLYLQMNSLRAEDTAVYFCARLGEGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG